(19) United States
(12) Patent Application Publication
(10) Pub. No.: US 2025/0308639 A1
(43) Pub. Date: Oct. 2, 2025

(54) METHOD AND SYSTEM FOR MODELING THE PHYSICAL EFFECTS OF SOLVENT AT THE MOLECULAR LEVEL

(71) Applicant: Artemis Discovery LLC, Philadelphia, PA (US)

(72) Inventor: Randy Jay Zauhar, Philadelphia, PA (US)

(21) Appl. No.: 19/097,226

(22) Filed: Apr. 1, 2025

Related U.S. Application Data
(60) Provisional application No. 63/572,480, filed on Apr. 1, 2024.

(51) Int. Cl. G16C 10/00; G16C 20/70

(52) U.S. Cl. CPC G16C 10/00; G16C 20/70

(57) ABSTRACT

This describes a new method for computing the effects of aqueous solvent at the molecular level, including energies, forces and dielectric screening, using the continuum approximation. The method provides a computer simulation of effects of solvent on a molecule by accessing a model of the molecule, defining a surface that corresponds to a boundary of solvent contact with the molecule, and partitioning the surface using discrete surface elements. The method also defines field points in the solvent near the surface elements of the molecule. The system uses the model, the surface elements and the field points to compute a distribution of polarization charge, and it measures mechanical effects on the molecule due caused by the polarization charge and by pressure exerted by the polarization charge. The system generates a visual representation that is output on a display device.

### CROSS-REFERENCE TO RELATED APPLICATIONS

This application claims priority under 35 U.S.C. 119(e) to U.S. Provisional Patent Application Ser. No. 63/572,480, filed on Apr. 1, 2024, which is hereby incorporated by reference in its entirety.

### BACKGROUND

Computing solvation effects for macromolecules remains a challenging and central problem in diverse areas of molecular modeling, biophysics, and computer-aided drug design. While using explicit solvent in conjunction with molecular dynamics or Monte Carlo simulation is the current standard for modeling solvent, this atomistic approach is problematic as it is computationally demanding and, at the same time does not lead to straightforward interpretation and insight. One rarely sees the effects of solvation being explicitly teased out in studies using explicit solvent, partly because the commonly-used Particle Mesh Ewald (PME) technique for long-range electrostatics does not support efficient decomposition of energies into distinct contributions, such as protein-solvent interaction. Instead, the focus is usually on overall changes in free energy as a function of state (such as ligand binding or residue composition).

On the other hand, the continuum approach, which models the solvent surrounding a molecule as a continuous and highly-polarizable medium, provides explicit and phenomenologically-coherent estimates of the effects of solvent in stabilizing charges and in screening charge interactions. These estimates are free of the noise inherent in simulation approaches and depend only on assumptions of molecular shape, dielectric constants, and molecular charge model, thus they are promising alternatives to compute and model solvation effects in large molecules. However, the current implementations of the continuum approach in wide use have significant drawbacks, some ignoring the detailed shape of the solvent-solute boundary, others supporting only limited use scenarios such as single-point energies.

This document describes new methods and systems directed to solving some of the issues described above, and/or other issues.

### SUMMARY

In one aspect, a method for providing a digital computer simulation of effects of a solvent on a molecule is provided. The method comprises accessing a model of a molecule, the model comprising a digital representation of a collection of atoms each with radius and electric charge; defining a surface that surrounds the molecule of the model and that corresponds to a boundary of solvent contact with the molecule, and partitioning the surface using discrete surface elements, wherein the surface elements provide a non-spherical surface on the molecule; defining a plurality of field points in the solvent near the surface elements of the molecule; using the model, the surface elements and the field points to compute a distribution of polarization charge induced on the surface by an electric field corresponding to interaction of the solvent with the electric field and consequent polarization of the solvent; measuring one or more mechanical effects on the molecule due caused by the polarization charge and by pressure exerted by the polarization charge; and generating a visual representation of the distribution of the polarization charge induced on the surface by the electric field corresponding to the interaction of the solvent with the electric field and consequent polarization of the solvent; and causing a display device to output the visual representation.

In some embodiments, the method may also comprise partitioning the surface comprises defining the surface elements to include or neighbor one or more of the atoms of the molecule. In some embodiments, the method may comprise measuring the one or more mechanical effects by a direct through-space force at each of the atoms due to the electric field caused by the induced distribution of polarization charge acting upon a charge associated with the atom. In some embodiments, the method may further comprise measuring a pressure exerted by polarization density defined on the surface elements.

In some embodiments, the method may include defining the field points by deriving the field points from spherical probes that are offset from and in contact with the surface or a regular grid exterior to the surface, or a combination of the spherical probes and the regular grid around the molecule. In some embodiments, the field points may comprise center of probes, and defining the probes normal to the surface elements at a separation distance equal to a solvent radius at about 1.5 Å. In some embodiments, the method may comprise measuring the pressure exerted by the polarization charge for each surface element. The method may comprise using a probe offset from the surface element to apply a pressure vector to the surface element, where the probe transfers contact forces to the atoms of the collection of atoms that the probe contacts. In some embodiments, the method may further comprise measuring total pressure exerted by the polarization charge on the atoms, by adding individual contact forces exerted on individual atoms by pressure of the surface elements.

In some embodiments, the field points comprise a grid, and the grid may comprise defining a series of voxelized volume elements exterior to the surface of the molecule, providing grid points at centers of the voxelized volume elements, and generating a layer of the grid points around the molecule using volume elements that adjoin the surface.

In some embodiments, measuring one or more mechanical effects may include determining one or more properties of a molecule: solvation energy, reaction force, reaction pressure, coulomb interaction, binding energy, charge state, ionization energy, interaction energy, or pKa.

In another aspect, computer program product comprising a memory device that stores programming instructions is disclosed. The program may be configured to cause a processor to provide a digital computer simulation of effects of a solvent on a molecule by initializing a model of a molecule, the model comprising a collection of atoms each with radius and electric charge, defining a surface that surrounds the molecule and that corresponds to a boundary of solvent contact with the molecule, and partitioning the surface using discrete surface elements, defining a plurality of field points in the solvent near the surface elements of the molecule, using the model, surface elements and field points to compute a distribution of polarization charge induced on the surface by an electric field arising from the fixed charges associated with the atoms, and corresponding to interaction of the solvent with the electric field and consequent polarization of the solvent, and measuring one or more mechanical effects on the molecule due caused by the polarization charge and by pressure exerted by the polarization charge; wherein the surface elements provide a non-spherical surface on the molecule.

In some embodiments, the programming instructions to partition the surface may comprise instructions to define the surface elements to include or neighbor one or more of the atoms of the molecule, and the programming instructions to measure the one or more mechanical effects comprise instructions to measure a direct through-space force at each of the atoms due to the electric field caused by the induced distribution of polarization charge acting upon a charge associated with the atom. In some embodiments, the programming instruction to provide the digital computer simulation may further comprise instructions to measure a pressure exerted by polarization density defined on the surface elements.

In some embodiments, the programming instructions to define the field points may comprise instructions to derive the field points from spherical probes that are offset from and in contact with the surface, a regular grid exterior to the surface, or a combination of the spherical probes and the regular grid around the molecule. In some embodiments, the programming instructions to measure the pressure exerted by the polarization charge may comprise instructions to, for each surface element, use a probe offset from the surface element to apply a pressure vector to the surface element, wherein the probe transfers contact forces to the atoms of the collection of atoms that the probe contacts.

In some embodiments, the programming instruction may further comprise instructions to measure total pressure exerted by the polarization charge on the atoms, by adding individual contact forces exerted on individual atoms by pressure of the surface elements.

In some embodiments, the field points of the computer program may comprise a grid and the programming instructions to define the grid comprise instructions to define a series of voxelized volume elements exterior to the surface of the molecule, provide grid points at centers of the voxelized volume elements, and generate a layer of the grid points around the molecule using volume elements that adjoin the surface.

In some embodiments, the programming instructions may further comprise instructions to generate a visual representation of the distribution of the polarization charge induced on the surface by the electric field corresponding to the interaction of the solvent with the electric field and consequent polarization of the solvent, and cause a display device to output the visual representation.

It should be noted that any feature described above may be used with any particular aspect or embodiment of the invention. The invention may be implemented as a device (e.g. a mobile device) configured to carry out any of the described methods.

### BRIEF DESCRIPTION OF THE DRAWINGS

FIG. 1 illustrates a flow chart of showing the protocol for simulating solvation effects for molecules using methods described in this document.

FIG. 2A illustrates discrete solvation of a molecule.

FIG. 2B illustrates continuum solvation of a molecule.

FIG. 3A illustrates grid-based continuum solvation of a molecule.

FIG. 3B illustrates surface-based continuum solvation of a molecule.

FIG. 4A illustrates an example of a field point in a solution near the surface of a molecule, as shown in stick structure.

FIG. 4B illustrates an example of a field point in a solution near the surface of a molecule, as shown in stick and electric field structure.

FIG. 4C illustrates an example of a field point in a solution near the surface of a molecule, as shown in electric field structure.

FIG. 5A illustrates a molecule with field points and associated distribution of induced polarization charge.

FIG. 5B illustrates a molecule with field points and associated distribution of induced polarization charge enclosed in a solvent-accessible surface.

FIG. 6A illustrates the method used to compute the mechanical pressure exerted on a molecule by the polarized solvent using probe radius on molecule surface.

FIG. 6B illustrates the method used to compute the mechanical pressure exerted on a molecule by the using probe pressure exerted on adjacent atoms.

FIG. 7A illustrates an example stick representation that the methods described in this document may generate and output on a display device.

FIG. 7B illustrates another example stick and surface representation that the methods described in this document may generate and output on a display device.

FIG. 7C illustrates an example visual representation that the methods described in this document may generate and output on a display device showing force vectors.

FIG. 7D illustrates another example visual representation that the methods described in this document may generate and output on a display device showing force vectors.

FIG. 8A illustrates an application of the methods described in this document to the dynamical simulation of molecules at 0 picoseconds.

FIG. 8B illustrates an application of the methods described in this document to the dynamical simulation of molecules at 10 picoseconds.

FIG. 8C illustrates an overlay of simulations of FIGS. 8A and 8B.

FIG. 9A illustrates an experimental structure for the protein molecule in complex with the compound Imatinib.

FIG. 9B illustrates an additional example compound having similar structures to Imatinib.

FIG. 9C illustrates the binding of Imatinib and the library molecules against the three-dimensional structure of the protein ABL2.

FIG. 10 illustrates an iso-contour graph of electric potential found using the methods disclosed here (solid lines) and the BEM (dashed lines).

FIG. 11A illustrates unit cell for a membrane system that includes the human Serotonin-2A receptor packed with lipid molecules shown from extracellular side (top) and intracellular side (bottom).

FIG. 11B illustrates unit cells of the system of FIG. 11A for an infinite membrane structure.

FIG. 11C illustrates the extracellular (top) and intra-cellular surfaces (bottom) generated for the membrane system of FIG. 11A.

FIG. 11D illustrates another view of membrane structure of FIG. 11A.

FIG. 12 illustrates example elements of a computing device that may be used to implement the methods or serve as a system embodiment.

### DETAILED DESCRIPTION

In this document, the singular forms “a,” “an,” and “the” include plural references unless the context clearly dictates otherwise. The term “comprising” (or “comprise” or “comprises”) means “including (or include or includes), but not limited to.” Unless defined otherwise, all technical and scientific terms used in this document have the same meanings as commonly understood by one of ordinary skill in the art.

In this document, terms that are descriptive of relative position such as “upper” and “lower”, “top” and “bottom”, “horizontal” and “vertical” and the like are intended to indicate relative positions with respect to the components for which those terms are descriptive, and are not intended to be absolute and require that the component remain in that absolute position in all configurations.

Except where specifically stated otherwise, numeric descriptors such as “first”, “second”, etc. are not intended to designate a particular order, sequence or position in an overall process or schema, but instead are simply intended to distinguish various items from each other by describing them as a first item, a second item, etc.

The terms “substantially” and “approximately”, when used in reference to a value, means a range that is within +/−10% of the value. When used in reference to a feature of an object, such as a substantially planar surface, terms such as “substantially” and “approximately” mean that the primary portion of the object exhibits the feature, although other portions may deviate. For example, a cleaning card in the form of a card from which embossments extend is considered to be a substantially planar surface.

The illustrative embodiment of the present disclosure enables a novel simulation of the effects of aqueous solvents at the molecular level, including energies, forces, and dielectric screening. The present disclosure uses a novel continuum-based approximation algorithm to enable the modeling of the solvent surrounding a molecule as a continuous and highly-polarizable medium. The present disclosure is thus particularly applicable to the simulation of solvation effects for macromolecules for a better understanding of biological processes at the molecular level with a significant reduction in computational resources and simulation duration.

The effects of aqueous solvents are essential to understanding the folded structure of proteins, interaction in protein complexes, as well as binding of small molecules (e.g., drugs and metabolites) to large macromolecules (e.g., proteins, nucleotides, and carbohydrates). As essentially all biological molecules include ionized/charged or polar chemical functional groups, the stabilization of these charged and polar groups maintains the three-dimensional structure of macromolecules and the organization of membranes and modulates the interactions between the molecules.

The physical effects of aqueous solvents can be modeled at the molecular level using a variety of methods and can reproduce experimental measurements such as the free energy of solvation of molecules. The continuum approach represents the aqueous solvent as a continuous medium that responds to the presence of charges by polarizing. The medium creates a reaction field that stabilizes the charges and screens, i.e., reduces, the magnitude of the interactions between them.

In the continuum model, the effects of solvent polarization can be completely described using a distribution of induced polarization charge defined on the surface that separates solute from solvent, which in the case of molecular modeling is typically defined by the solvent-accessible surface. This document proposes an alternative method to compute the density of induced polarization as a function of molecular shape and distribution of the fixed charges of the molecule. It provides for the rapid update of computed effects corresponding to new configurations of the molecular charge distribution, is relatively insensitive to irregularities in the solvent-accessible surface, and is less computationally demanding than prior methods.

Thus, the disclosed method may allow for the computing of the total force due to solvent polarization on each atom of the model, by summing the negative gradient of the interaction between each atom and the induced polarization charge on the surface as a function of atomic position and adding the pressure force due to polarization charge measured for each atom. Furthermore, the present method may allow the application of dynamic simulation of one or more molecules in the presence of aqueous solvent, where the forces due to solvent polarization are computed for the molecules being simulated and combined with non-solvent contributions such as direct electrostatic and van der Waals interactions within and between the model molecules, obviating the need to include a large number of explicit solvent molecules to introduce the effects of aqueous solvation.

Further, the present method may allow for determination of optimal conformations of flexible molecules, and configurations of interacting molecules, where the induced polarization charge is computed for the model molecules and used to compute the solvation energy as a function of the conformation of the molecules and/or their interacting configuration, with the solvent contribution typically being combined with other distinct non-solvent contributions as found using existing methods of molecular mechanics.

Due to the high dielectric constant of water, the effects of aqueous solvent polarization on a solute molecule are closely similar to those expected of a metallic conducting substance, for which the dielectric constant is essentially infinite, and inside of which the electric potential is zero. In the approach described in this document, a computer-implemented simulation models a molecule in solvent and defines various “field points” in the exterior (on the solvent side) of a triangulated molecular surface. The methods adjust the vector of induced charge density (taken as constant over each surface element) so as to drive the potential to zero at the external points. As used herein, the “surface element” refer to the discrete segment or a grid used to approximate a boundary on molecule's surface where the governing equations/calculations are applied. As used in this document, the surface elements may provide a spherical or non-spherical surface on the molecule, including but not limited to triangles, quadrilaterals or higher-order polygonal shapes. This process is carried out using a least-squares algorithm, leading to an external potential which is zero at the field points, and a total countercharge (surface integral of the induced charge) which is very nearly equal and opposite to the total fixed charge, as electrostatic theory requires.

While only directly providing the conducting solution, the solutions described in this document compute an estimate of the solution for finite solvent dielectric constant using a perturbation approach which is exact for spherical boundaries, and which offers reasonable accuracy even when the charge is significantly off-center. In practice, fixed charges near the surface effectively see a local geometry that is mostly spherical, corresponding to the radius of the surface atom to which the charge belongs.

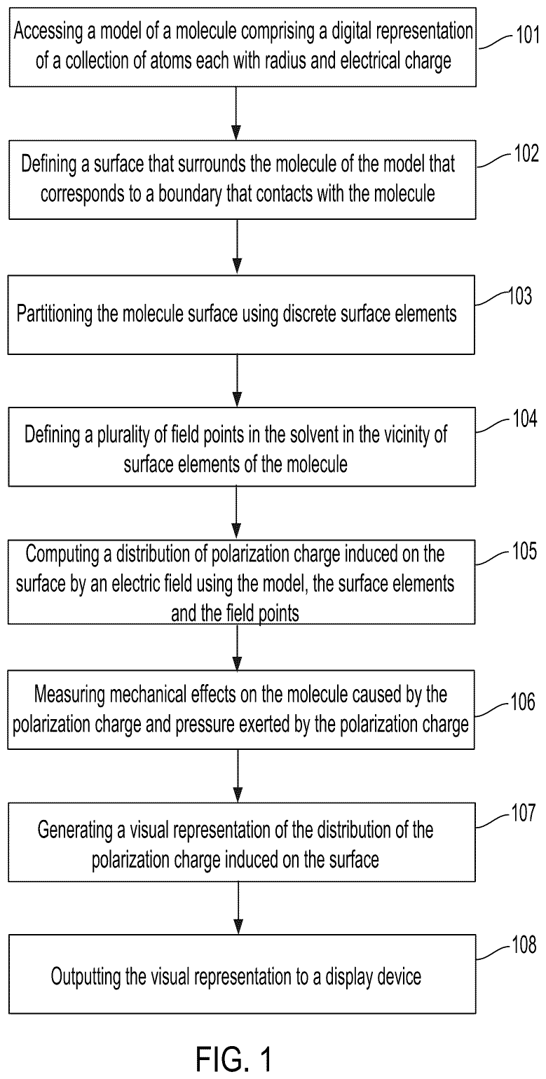

The disclosed method provides a digital computer simulation of the effects of a solvent on a molecule. Referring to FIG. 1, the method may include a computer-implemented method of modeling a molecule by accessing a model of a molecule comprising a digital representation of a collection of atoms each with defined radius and electrical charge 101; defining a surface that surrounds the molecule of the model and that corresponds to a boundary of solvent contact with the molecule102; partitioning the surface using discrete surface elements 103, wherein the surface elements provide a non-spherical surface on the molecule; defining a plurality of field points in the solvent near the surface elements of the molecule 104; using the model, the surface elements and the field points to compute a distribution of polarization charge induced on the surface by an electric field corresponding to interaction of the solvent with the electric field and consequent polarization of the solvent 105; measuring one or more mechanical effects on the molecule due caused by the polarization charge and by pressure exerted by the polarization charge 106; generating a visual representation of the distribution of the polarization charge induced on the surface by the electric field corresponding to the interaction of the solvent with the electric field and consequent polarization of the solvent 107; and causing a display device to output the visual representation 108.

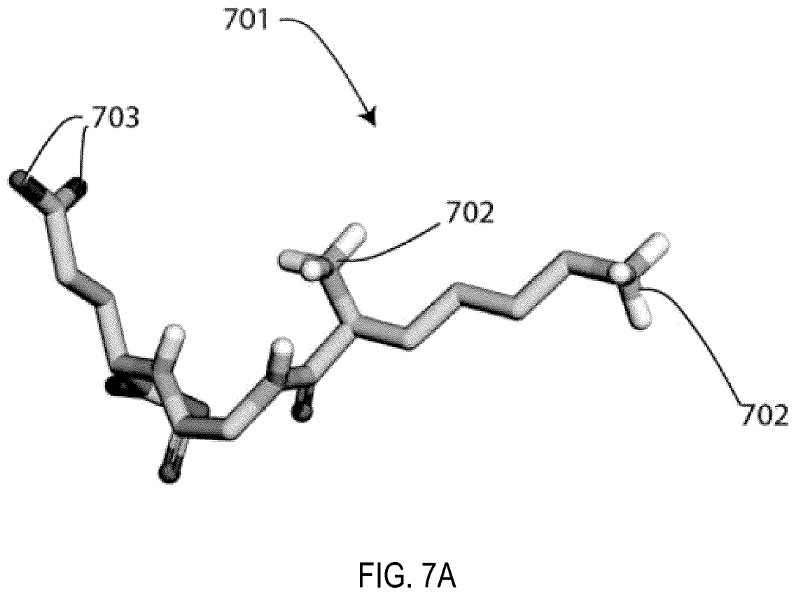

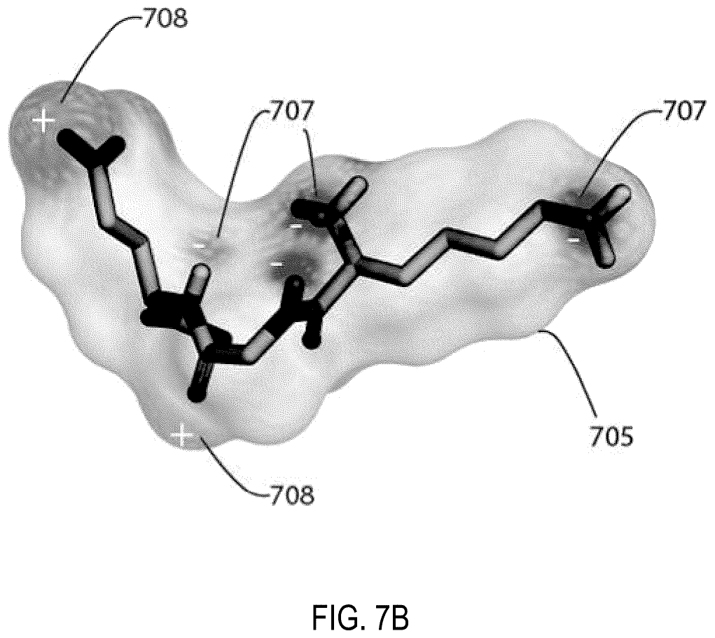

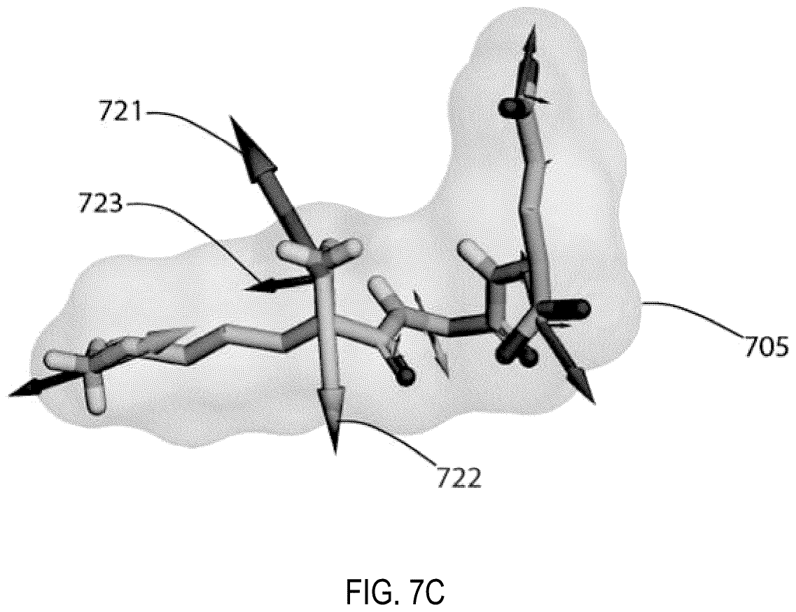

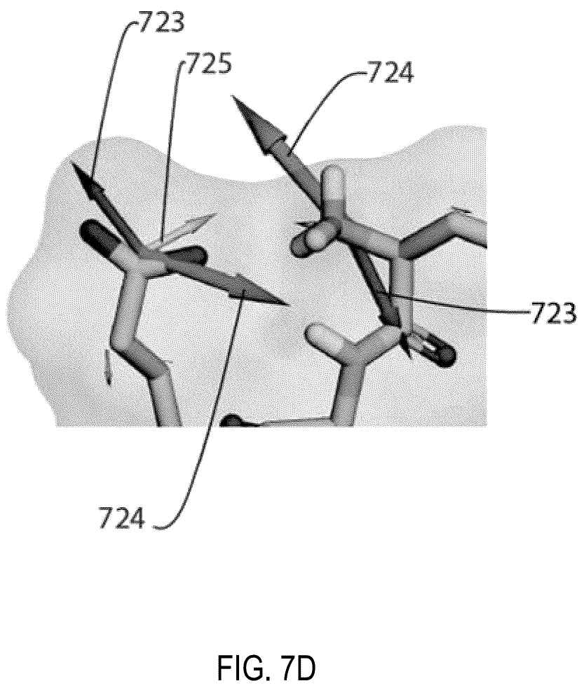

The method is used to analyze a molecule wherein a solvent-accessible surface provides a boundary that closely follows the three-dimensional configuration of atoms and is partitioned into discrete elements. The methods may also include generating a visual representation of the distribution of the polarization charge induced on the surface by the electric field corresponding to the interaction of the solvent with the electric field due to the distribution of molecular charge, and consequent polarization of the solvent, along with causing a display device to output the visual representation. Embodiments of visual representations that may be outputted to a display device are presented in FIGS. 7A through 7D, which are further described below.

In various embodiments, the induced polarization charge exerts mechanical forces on the molecule via two mechanisms, one of these being a direct, through-space interaction that is defined as the reaction force, and the other a reaction pressure, which arises from a normal pressure exerted by the polarization of the solvent, and which is described subsequently. The through-space reaction force on atom k with position vector Rₖ is found using Coulomb's law, and depends on the atomic charge of the atom qₖ, the discrete elements that comprise the surface (each with centroid at position vector C_{α} and with area A_{α} in the embodiment described here), and the computed distribution of induced polarization charge, which may be expressed as a density σ_{α} (measured as elementary charge per Å² in this embodiment) for each element. Given this terminology the reaction force at atom k is then computed exactly in cgs units as:

$${\vec{F}}_{k}^{react} = {q}_{k} {\sum }_{α = 1}^{n} ( {σ}_{α} {A}_{α} ) \frac{( {\vec{R}}_{k} - {\vec{C}}_{α} )}{{\vert {\vec{R}}_{k} - {\vec{C}}_{α} \vert}^{3}} \qquad ( 1 )$$

where the sum is taken over the n elements that comprise the surface separating the molecule from the solvent. In this expression, the product σ_{α}A_{α} is the total induced polarization charge on element α, and the equation corresponds to the vector form of Coulomb's inverse-square law for electrostatic force. A related scalar expression is used to compute the electric potential energy due to solvation measured at the same atom:

$${E}_{k}^{react} = {q}_{k} {\sum }_{α = 1}^{n} ( {σ}_{α} {A}_{α} ) \frac{1}{\vert {\vec{R}}_{k} - {\vec{C}}_{α} \vert} . \qquad ( 2 )$$

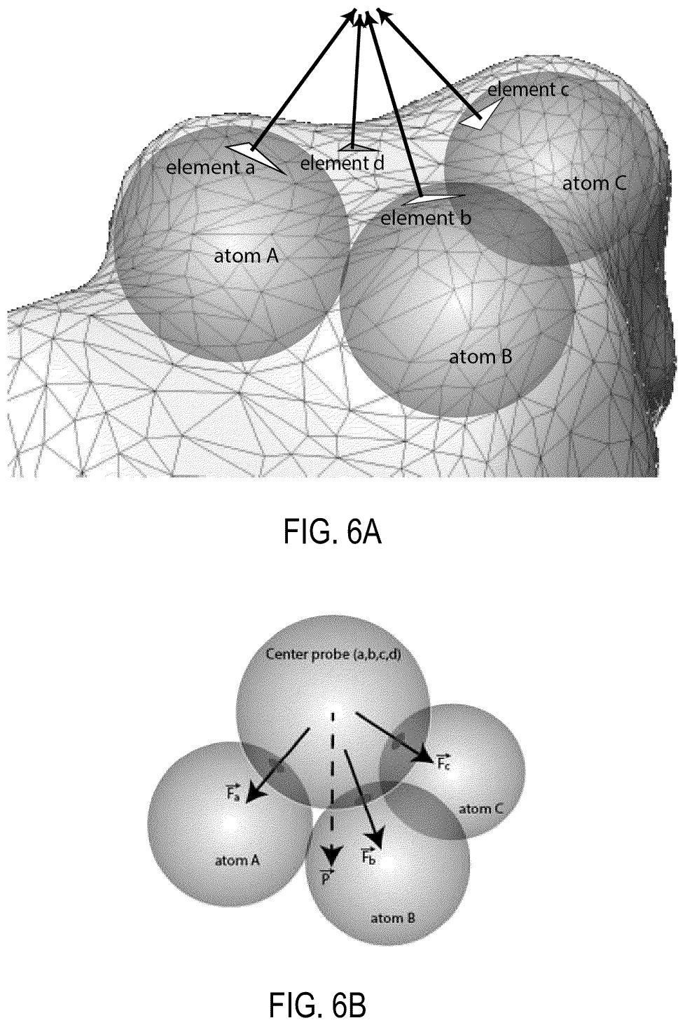

In various embodiments the reaction pressure, which has position-dependent magnitude 2πσ² over the surface, and with the force being directed always normal to the surface and inward toward the molecule is computed. This is a contact force that will only be experienced by atoms that directly define the surface, i.e. that are modeled as in contact with solvent. Some surface elements are associated with only one atom, and their pressure force can be directly transferred to that atom, but many elements are in “reentrant regions” that span more than one atom, and it is necessary to construct a general modelling procedure to define the transfer of pressure from surface elements to the atoms. As illustrated in FIGS. 6A and 6B, this embodiment assumes a solvent-accessible surface, where each surface element is associated with a “probe,” a sphere offset from the surface with radius appropriate for a water molecule (˜1.5 Å), and which makes contact with the molecule at points on one, two or three atoms. Next, the pressure force of the surface element at the center of this probe is applied, and the assumption of mechanical equilibrium of the probe then uniquely defines the contact forces with the atoms. The pressure force for each surface element is thus converted into contact forces with the atoms associated with the element. The contact forces for each atom are summed to produce a net reaction pressure force for that atom. The reaction pressure on an atom will be identically zero if it is not solvent-exposed, i.e. has no associated surface elements.

FIG. 6A illustrates four surface elements, a, b, c, and d, with surface normal extended by the probe radius, defining the center of the probe. In various embodiments, each surface element has a reaction pressure vector in the direction opposite the normal to the probe. The exerted pressures of surface elements a, b, c, and d, are defined as vector pressure contributions Pₐ, P_{b}, P_{c}, and P_{d}, which sum to yield a total pressure P (shown as dashed arrow \vec{p}).

In various embodiments, this pressure P may be converted to contact forces Fₐ, F_{b}, and F_{c} exerted on the three atoms A, B, and C that the center probe touches as seen in FIG. 6B. To ensure the static equilibrium of the probe, the total reaction pressure vector \vec{p} may be defined as:

$${\to }_{P} = - {F}_{a} {\hat{u}}_{a} - {F}_{b} {\hat{u}}_{b} - {F}_{c} {\hat{u}}_{c} \qquad ( 3 )$$

where unit vectors û_{a,b,c} are directed from the probe center towards the surface atoms. Accordingly, by applying the dot product to each unit vector in equation (3), contact force magnitudes may be expressed as equation (4) below, where ûₓ=ûₐ, û_{b}, or û_{c}

$${\to }_{P} \cdot {\hat{u}}_{x} = - {F}_{a} {\hat{u}}_{a} \cdot {\hat{u}}_{x} - {F}_{b} {\hat{u}}_{b} \cdot {\hat{u}}_{x} - {F}_{c} {\hat{u}}_{c} \cdot {\hat{u}}_{x} \qquad ( 4 )$$

In various embodiments, equations (3) and (4) may be applied when there are one, two, or three atoms in contact with the center probes. In various embodiments, when the system comprises more than three contact atoms with the center probe, two alternative approaches may be utilized to compute the solvent contribution to the electrostatic potential and forces: (1) take the least square solution of the minimum forces that satisfy the equilibrium; or (2) apply equations (1) and (2) with three contact atoms that are closest to the probe.

In various embodiments, the field points, which comprise the key feature that distinguishes this invention from conventional approaches, are placed at positions on the solvent side of the surface enclosing the molecule. The number and positions of the field points are not explicitly defined, the assumptions being only that they are in the immediate vicinity of the molecule, and provide a uniform sampling of the solvent volume near to the molecule. While numerical results are affected by the number and physical distribution of field points, their specific placement is arbitrary and can be achieved using diverse procedures. In various embodiments, field point placement can be carried out using a selection of the same spherical probe positions used to model the reaction pressure, as just described. Since available probe positions are far in excess of the number of field points necessary for an accurate computation of solvation effects, the positions can be randomly down-sampled, or alternatively neighboring probes can be grouped into clusters, and field point position selected from the cluster centers. Various embodiments can utilize a totally distinct procedure to assign field point positions, wherein a defined volume that includes the molecule is partitioned into regular volume elements using the well-known method of voxelization. Voxelization uses the existing solvent-accessible surface to label volume elements as interior or exterior to the surface, and the centers of volume elements on the exterior (solvent) side are then available to be assigned as field points. These points can be down-sampled to reduce their number, or subject to other constraints, such a distance from the solvent-accessible surface, to control their selection. Field points generated by any number of distinct procedures can be used separately or in combination. In various embodiments the use of field points derived from probe spheres may be found to provide a thorough and reliable coverage of the volume near to the surface of the molecule, while field points derived from the voxelized volume may provide a convenient route to placing field points that are regularly-spaced and further from the molecular surface. Using this approach, it is possible to generate a layer of grid points around the molecule that adjoin the surface, which can either replace or supplement field points derived from probes.

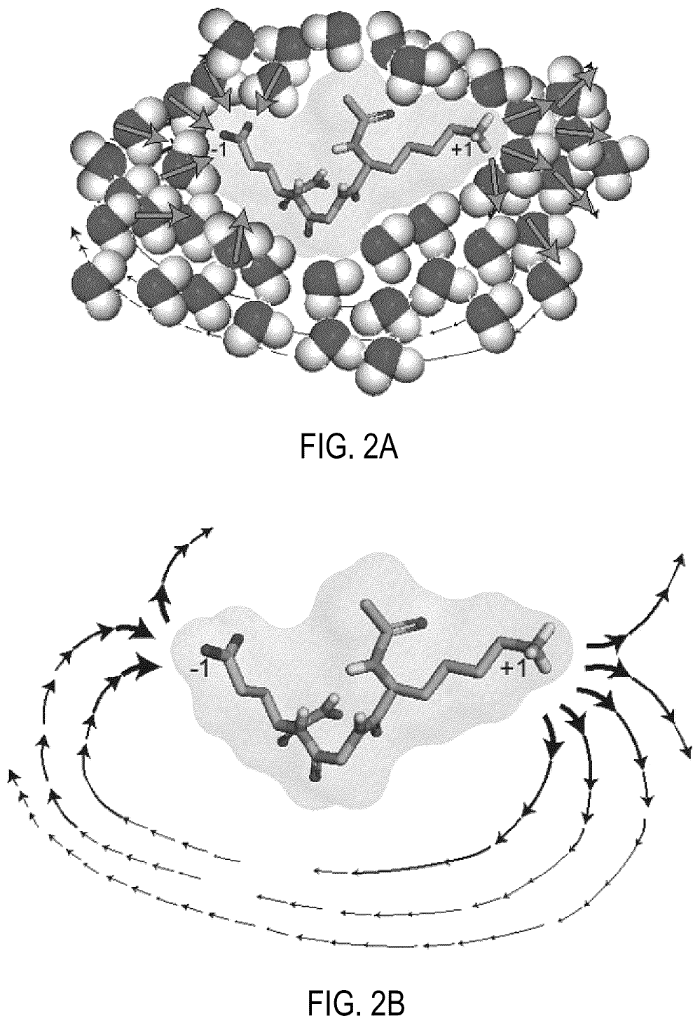

In the continuum method of the present disclosure, solvent is not represented by discrete or individual molecules, but rather as a continuous polarizable medium. Conversely, the solvated molecule of interest, e.g., proteins and small molecule binding partner, are defined at an atomic level, and the shape of the cavity that encompasses the solvated molecule is represented using a well-defined geometry, typically provided by a solvent-accessible surface. FIGS. 2A and 2B illustrate discrete (FIG. 2A) and continuous/continuum (FIG. 2B) representations of solvation on a small peptide molecule with positively charged and negatively charged amino acid termini.

In the discrete solvent view as shown in FIG. 2A, individual/discrete water molecules near the peptide molecule are associated with a time-averaged orientation, i.e., permanent dipole moments in the individual solvent molecules, e.g., H₂O molecule, as indicated by the arrow, point toward a negative terminus, and away from a positive terminus of the peptide molecule. In the continuum system as illustrated in FIG. 2B, the oppositely charged termini induce a vector field of polarization in the solvent medium, and a volume element at any position has an associated induced dipole moment. While both discrete and continuum methods may be used to observe solvation effects on macromolecules, the discrete method requires dynamical simulations which require significant computational resources. Accordingly, the present disclosure provides a novel method to address such technical limitations by utilizing the continuum solvation method to elucidate solvation effects for biomolecules, and for solute/solvent systems in general.

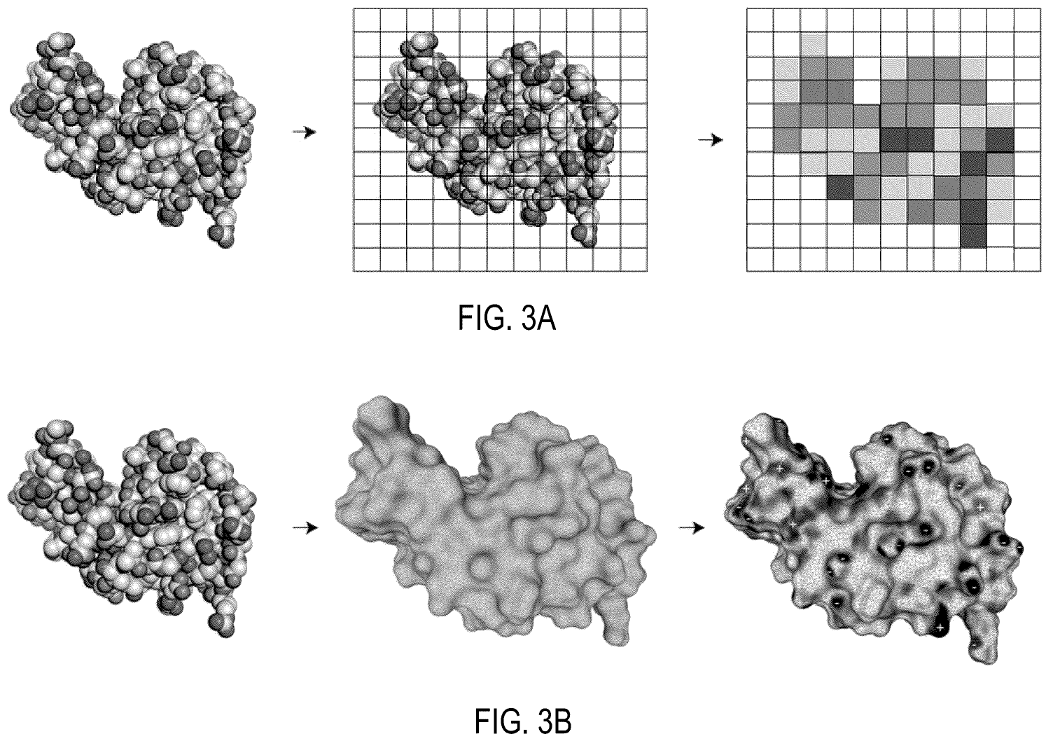

The continuum method presents two distinct, mathematically complementary approaches to solve for and compute the effects of solvent polarization. In “volume-based” approach, the electrostatic effects of polarization are explicitly computed at each point in space, expressed mathematically as a vector field. While analytic solutions using this approach can be found for molecules of simple shape (e.g. ellipsoids), for molecules with realistic shape, a numerical solution procedure is required, as illustrated in FIGS. 3A and 3B. FIG. 3A illustrates a “volume-based” approach to numerical solution, wherein the macromolecule and a portion of the surrounding solvent are divided into small cubes using a regular grid. In various embodiments, the fixed charges, e.g., the full charges of ionized functional groups of the macromolecule, are distributed over the grid. The grid cubes in the molecule interior are assigned low polarizability, corresponding to dielectric constant between one and four, while the cubes in the exterior solvent are assigned a larger dielectric constant about 80, indicating high polarizability. Afterwards, the total electric potential is computed at each grid element using an iterative solver. While mathematically correct, this approach is difficult to use in many practical applications, as the electric potential computed necessarily mixes the contributions of the fixed atomic charges of the molecule and the polarization of the solvent. Since the former contribution increases without limit in the vicinity of an atomic charge (if modeled as a point charge), special methods are needed to disambiguate the contribution of solvent polarization. Given the coarse partitioning required in practical computations it is not possible to recover accurate estimates of solvation forces and pressures, precluding the use of the volume-based approach in most applications.

The alternative “surface-based” approach, which provides the physical basis of this disclosure, is illustrated in FIG. 3B. Here, a surface separates the molecular interior and the external solvent. The surface is partitioned into discrete surface elements to support numerical computation. Given well-known results of physics, there exists a distribution of induced polarization charge on the surface, the electrostatic effects of which exactly reproduce the effects of the polarization field computed in the volume approach. This distribution is visualized in FIG. 3B as shaded density on the surface, which varies in intensity and polarity as a function of position on the surface. Here, the polarity in localized regions is indicating using + or −, indicating the presence of positive or negative polarization charge respectively. In contrast to the volume-based approach, surface methods enable the immediate computation of energies, forces and pressures at the atomic level, since the effects of solvent polarization are directly computed from the induced surface charge distribution, which is physically separate from the atomic charges. This opens the door to many practical applications.

For practical computations for molecules of arbitrary shape, applying the surface-based approach requires introducing a numerical procedure. One existing numerical method for the surface-based approach is the Boundary Element Method (BEM). While mathematically correct, the BEM is prone to numerical instability if the solvent-accessible surface is not smooth, in particular, if it includes sharp edges or wrinkles, artefacts that are often generated by the algorithms that compute molecular surfaces. Moreover, the BEM requires computing and storing all pairwise interactions between surface elements and effectively solving a large system of simultaneous equations, the size of which scales with the number of surface elements, leading to challenging computations if large molecules are to be handled. These difficulties have limited the adoption of the surface-based approach, despite its clear superiority in practical application.

The illustrative embodiment of the present disclosure provides alternative to conventional BEM to efficiently resolve the surface charge distribution on solute molecules of any size, but is especially pertinent in application to large biomolecules such as proteins where the computational demands are greatest. The BEM for solving induced surface charge requires a very large set of coefficients corresponding to every pairwise interaction between the elements of the molecule surface. In contrast, the disclosed method is dependent upon the placement of a limited number of field points in the exterior, solvent volume, the required number of which is much smaller than the number of surface elements. If n is the number of surface elements and m the number of field points, this means that a computation of order n×n is immediately reduced to m×n, with m<<n. In a further mathematical development presented subsequently, it is found possible to further reduce the computational order to m×m, providing a dramatic reduction in computational resources required. In various embodiments, the number of field points m may be about 10- to 20-fold fewer than the number of surface elements n, implying a maximum computational speed-up of 100- to 400-fold relative to the conventional BEM.

Moreover, while the conventional BEM method is known to be mathematically rigorous, the use of field points as disclosed here, is more akin to various quadrature methods used to fit mathematical functions to meet defined constraints, which affords a less computationally demanding approach. The presently disclosed method introduces the concept of field point basis functions, which are the distributions defined over the entire surface of the molecule and which individually set the potential to zero at these field points. The field point approach provides an immediate improvement in efficiency without compromising the accuracy and precision of the calculation.

In computing discretized representation of induced surface charge via the field point approach, two strategies may be utilized for solving the optimal distribution of induced charge on a molecule surface. First, in Full Matrix Solution (FMS) approach, (i) a vector Q of fixed charges with length N_{Q}, (ii) a polyhedral surface defined by vectors of element areas A and element centroids C each with of length n, and (iii) a vector F of m field point positions exterior to the surface, are used as a model system to compute an n-vector of polarization charge densities σ for the surface elements. Assuming constant density over each element, matrix D^{Q} is computed such that the vector E_{Q}^{F} of the electric potential as shown in equation (5):

$${E}_{Q}^{F} = {D}^{Q} Q \qquad ( 5 )$$

and D^{Q} is further defined as:

$${D}_{α j}^{Q} = \frac{1}{\vert {\vec{R}}_{F}^{α} \to {\vec{R}}_{Q}^{j} \vert} = \frac{1}{{d}_{j}^{α}} , \qquad ( 6 )$$

with field point α at position R_{F}^{α} and j^{th} fixed atomic charge at position R^{j}_{Q}. Accordingly, the potential at the field points due to the surface charge distribution is defined as:

$${E}_{S}^{F} = {D}^{S} ( \begin{matrix}{A}_{1} & & \\ & ↘ & \\ & & {A}_{n}\end{matrix} ) \vec{σ} = K \vec{σ} , \\ with \qquad ( 7 )$$

$${D}_{α j}^{S} = \frac{1}{\vert {\vec{R}}_{F}^{α} - {\vec{C}}_{j} \vert} = \frac{1}{{r}_{j}^{α}} , \qquad ( 8 )$$

where Cⱼ is the centroid of the j^{th} surface element and A is a diagonal matrix of the surface element areas. Accordingly, E_{S}^{F} in equation (7) may be reduced to:

$${E}_{S}^{F} \vert \vec{σ} = {\vec{E}}_{Q}^{F} \qquad ( 9 )$$

which can be efficiently solved by several approaches such as standard least-squares solver. This approach provides a minimum-norm solution that is exact, in the sense that the residual error is reduced essentially to zero. This provides a solution for the assumption of the conducting case (infinite exterior dielectric constant) and represents a computation of order n×m.

Next, in some embodiments, Reduced Matrix Solution (RMS) approach using basis vectors may be utilized for solving for the optimal distribution of induced charge on a molecular surface. RMS relies upon the mathematical development that follows, which forms part of this disclosure. For a given estimate of the induced surface charge density σ′, the vector of signed errors over the field points is defined as:

$$\vec{err} = K {\vec{σ}}^{′} + {E}_{Q}^{F} \qquad ( 10 )$$

where Kσ′ provides the electric potential due to the current estimate of the induced polarization charge at the field points, and

$${E}_{Q}^{F}$$

is the vector of the electric potential at the field points due to the fixed charges. Accordingly, the signed error will be non-zero if the solution σ′ does not exactly nullify the potential due to the fixed charges, as measured at the field points.

In various embodiments, for a single field point α, the associated error errⁿ may be distributed over the surface elements, weighting the assignment for each surface elements by a power p of the product of its area and inverse distance. Accordingly, for the j^{th} surface element and power p, the assigned error may be defined as:

$${err}_{j}^{α} = \frac{{{err}^{α} ( \frac{{A}_{j}}{{r}_{j}^{α}} )}^{p}}{{\sum }_{k = 1}^{n} {( \frac{{A}_{k}}{{r}_{k}^{α}} )}^{p}} = \frac{{{err}^{α} ( {K}_{α j} )}^{p}}{{K}_{\sum α}^{( p )}} . \qquad ( 11 )$$

Here rⱼ^{α} is defined as the distance between the element centroid and the field point, and that the area/distance (A/r) ratios are the elements of the coefficient matrix K, which was computed in equation (7). The sum of elements in row a of K, each raised to power p, is defined as K^{(p)}_{Σα}, and the full vector is defined as K^{(p)}_{Σ}. This assigned error can be immediately translated into a contribution to the induced charge density for the element, by inverting the sign of the error:

$$Δ {σ}_{j}^{α} {\vert}_{p} = - {( \frac{{A}_{j}}{{r}_{j}^{α}} )}^{- 1} {err}_{j}^{α} {\vert}_{p} = - {K}_{α j}^{- 1} \cdot {err}_{j}^{α} {\vert}_{p} . \qquad ( 12 )$$

It should be noted that in equation (12), K⁻¹ _{αj}, is simply the reciprocal of a coefficient from the matrix K, not an element of the inverse matrix. Next, the error at the selected field point may be updated by adding this contribution to the original solution as shown in equation (13), where it is seen that the error at the selected field point is now set to zero:

$$\begin{matrix}{err}^{α} \vert {Δσ}^{α} = {\sum }_{j} ( {K}_{α j} \cdot ( {σ}_{j}^{′} + {Δσ}_{j}^{α} ) ) \\ = {err}^{α} + {\sum }_{j} ( - {K}_{α j} \cdot {K}_{α j}^{- 1} \cdot {err}_{j}^{α} ) = 0.\end{matrix} \qquad ( 13 )$$

Accordingly, for each field point α, a series of solution vectors, that corresponds to p=1, 2, 3, . . . may be calculated, any of which set the error to zero for that specific field point. For a given p, solution Δσ^{∝} may be used as a basis for a least-squares solution that minimizes the mean error over all of the field points. For example, when p=1, the solution for each field point is constant over the surface while as p increases, the basis functions become more sharply localized in the vicinity of the field point.

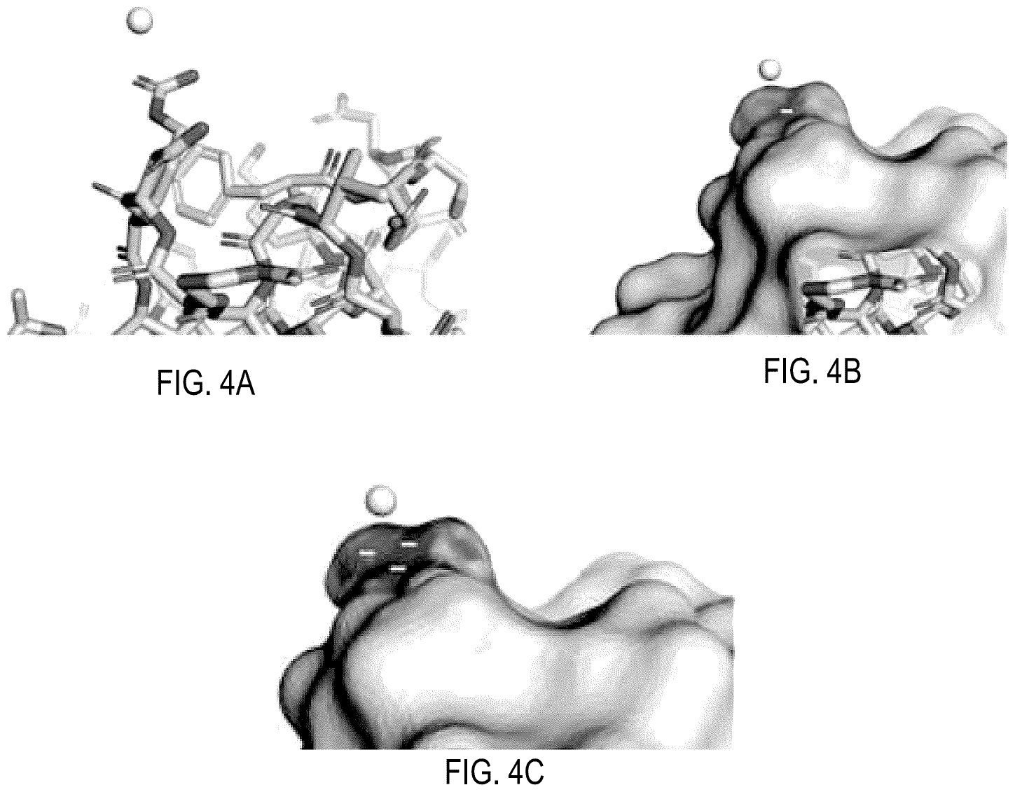

FIGS. 4A-4C illustrate basis functions for a single field point (small sphere) near amino acid residue Arginine 114 in a zoomed-in section of the protein Lysozyme (FIG. 4A). Basis solution vectors for exponent p=2 (FIG. 4B) and p=3 (FIG. 4C) are shown as negative density (−) on the surface, which arises in response to the positive charge of the ionized amino acid, and is localized in the immediate vicinity of the field point. As illustrated in FIGS. 4B and 4C, as exponent p increases, the surface charge distribution becomes more focused as the surface charge density becomes localized on the protein surface in the vicinity of the field point.

Accordingly, for a starting solution σ′ (which may be the zero vector) and a choice of p, a matrix Δσ (m×n) may be calculated from the current error, with each of the m computed using equation (12). This operation is economical as it involves only the element-wise power of the starting coefficient matrix K, along with row-sums of the power matrix. From Δσ, the matrix ΔErr of the shift in error at each of the m field points due to any one of the m basis solution may be calculated using equation (14) which defines the shift in error at field point α due to the basis solution associate with field point β.

$$Δ {Err}_{α}^{β} = {\sum }_{j} {K}_{α j} Δ {σ}_{j}^{β} . \qquad ( 14 )$$

Finally, a linear combination of the basis functions may be calculated to minimize the least-square error over all of the field points. Accordingly, for a given field point, the resulting error is a linear combination of signed errors associated with all m basis functions. Given an m×1 vector of coefficients C, the vector of error shifts may be defined as:

$$Δ \vec{err} ( C ) \to = Δ Err \cdot \vec{C} . \qquad ( 15 )$$

Accordingly, by solving the reduced m×m system to minimize the error, where ΔErr is the coefficient matrix, \vec{c} is the solution vector, the updated solution is:

$${\vec{σ} = {\vec{σ}}^{′} + ( Δ {σ}^{T} \cdot \vec{C} ) \vert}_{p} \qquad ( 16 )$$

where the solution depends upon exponent p.

Referring back to FIGS. 4B and 4C, the basis solutions become more sharply localized as p increases which indicates a recursive procedure, where an initial solution set to zero, and the contribution of the optimal constant solution (i.e., p=1) is added, and subsequent contributions at higher exponent p is added as illustrated in equation (17).

$${{\vec{σ}}_{p} = {{\vec{σ}}^{′}}_{p - 1} + ( Δ {σ}_{p - 1}^{T} \cdot {\vec{C}}_{p - 1} ) \vert}_{p} . \qquad ( 17 )$$

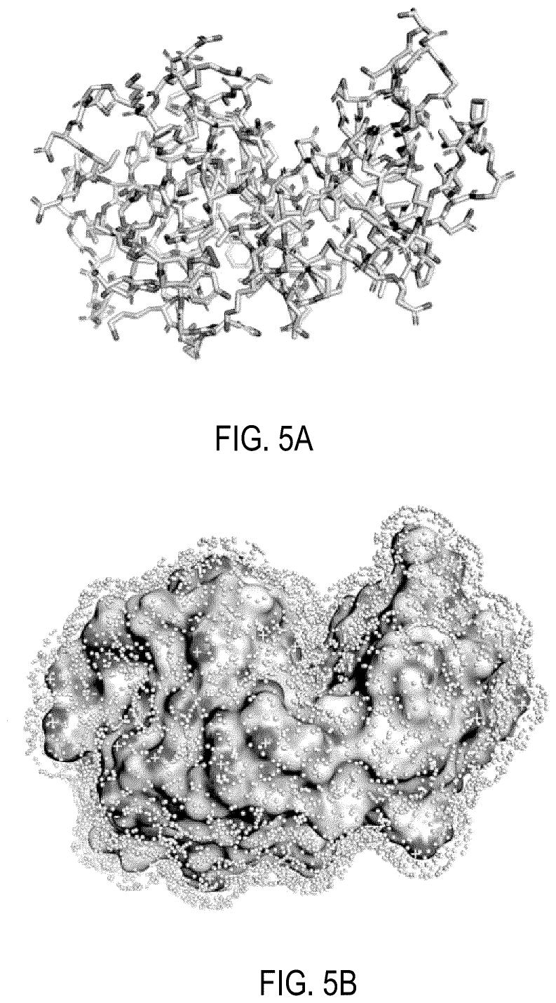

In practice, it is found unnecessary to carry out the series expansion implied by equation (17). Instead, the use of a single exponent p=2 provides a good numerical solution with very small least-squares residual, and the order of the RMS computation is thus m×m where m is the number of field points. FIG. 5A illustrates the entire protein Lysozyme, and FIG. 5B shows the protein enclosed in a solvent-accessible surface and 10,000 field points. The complete RMS solution for the induced polarization charge using exponent p=2 is shown as shading of the surface, which varies in intensity and polarity with position; the polarity in localized regions is indicated by + and − signs.

Once a solution of a has been obtained for one configuration of fixed molecular charges, solutions for other configurations of charges are inexpensive to compute, so long as the molecular surface does not need to be modified. This is because the result of the most demanding phase of the computation, Singular Value Decomposition (SVD), can be reused for multiple “right-hand sides” of the system of equations, corresponding to different vectors ΔErr in equation (15). This provides an important advantage for practical application, as discussed further below.

The solvent-accessible molecular surfaces used here are in defined in various embodiments using a small spherical “probe” of size appropriate for a single solvent molecule, corresponding to a radius of about 1.5 Å, and the points of contact between the probe and the atoms of the molecule define the boundary between the molecular interior and exterior solvent. In various embodiments, field points may be generated by the use of such spherical probes. The elements that define the surface, i.e., surface elements, all have associated normal with respect to the surface, which define a probe positioned from the center of the element using the assumed probe radius, defined as the “center probes”. In various embodiments, the positions of these center probes may or may not perfectly align with a geometric probe position defined by precise, physical contacts between the spherical probe and one or more atoms. For instance, given the atomic radii and the probe radius used to generate the surface, the probe may be in contact with an atom if the separation of the probe and atom centers differs from the sum of probe and atom radii by a small threshold, i.e., about 0.15 Å by default). In various embodiments, the center probe may have a solvent radius of about 1.5 Å. In various embodiments, each center probe may simultaneously be in contact with about one to three surface atoms. In various embodiments, a total pressure force associated with each probe at the center may be calculated as the sum of the pressure force vectors of all surface elements associated with the center probe. FIGS. 5A and 5B illustrate the protein molecule Lysozyme, with FIG. 5B illustrating a defined surface around which many field points are placed near the vicinity of the molecule surface, using a random selection of probe positions.

Applications of the forces and energies and other results computed by the methods described herein including, for example: prediction of the affinity and potency of drugs and metabolites binding to protein and DNA molecules, thus identifying therapeutic compounds with improved efficacy; determining the optimal configurations of interacting biological molecules such as the components of multimeric proteins, viral capsids and other biological complexes, thereby predicting their function in health and disease; simulating the biological effects of drug and hormones when forming complexes with cognate protein targets, including simulation of allosteric effects that determine drug action and potency; prediction of bioavailability of drug molecules and their disposition in vivo; prediction of solvent effects in synthetic materials such as nanoparticles leading to improved materials design; and any application which requires understanding the influence of solvent on behavior, function or properties.

The methods described in this document can allow for fast and accurate computation of relatively large proteins on inexpensive server hardware compared to conventional methods. The described method is robust to small perturbations in the surface geometry and provides accurate and precise results that can be applied in numerous problem domains, most immediately those involving optimization and simulation of biomolecular systems, and integration with and augmentation of various existing approaches for: predicting bioactivity of metabolites and drug-like molecules; predicting ionization states of chemical groups; computing interactions between biomolecules and other molecules or ions in solution; and estimating physico-chemical properties of molecules, such as bioavailability.

### Examples

In various tests of the present methods, simulations have been run on molecules that consider approximately 200,000 surface elements and 20,000 external field points. However, the invention is not limited to these parameters; any number of surface elements and field points may be considered and will be within the scope of this disclosure.

### Example 1

Comparison between the present embodiments and BEM established knowledge for a system with simple geometry, consisting of a spherical interface separating a region of low polarizability from external solvent, and containing a single fixed charge at specified radius. This is a common comparative example, as exact solutions are well-known.

The method described in this document was used to compute the solvation energy, interior potential, and exterior potential for a spherical interface containing a fixed unit charge at specified radius. The results were compared with the conventional, more computationally intensive BEM method. The interface consisted of a uniform spherical boundary of 10 Å radius, centered at the origin, and triangulated using a subdivision procedure that produced meshes with varying densities of elements, this done so that computations of progressively greater precision could be carried out, and numerical convergence of results tested. The sphere was initially subdivided into either 1,280 or 5,120 triangular surface elements, followed by either one or two rounds of subsequent subdivision of elements closest to the fixed charge, this procedure yielding final element counts that ranged from 1,280 to 13,408. The system included a single unit positive charge positioned at radius 4 or 8 Å along the z-axis.

For the present solution, field points were generated at 1 Å displacement from the surface elements centered along the outward-directed normal. The number of field points was set at ½ the number of surface elements up to a maximum of 6,000 and were placed at a random sample of available positions, with probability weighted by the normal field intensity of the fixed charge. The field point approach was carried out using the full matrix approach for these tests. The total induced polarization charge was always very close to −1, with deviations of +/−0.0002 charge unit or less.

Tables 1 and 2 summarize the results for both the present solution (CON) and the BEM approach for conducting molecule exterior, for charge radius of (a) 4 Å and (b) 8 Å. Using the present method, the solvation energies, reaction force, and reaction pressure converge to the exact/experimental values for the 4 Å radius system and converge to within 0.01 kcal/mol for the solvation energy and 0.02 kcal/mol/Å for the reaction force and reaction pressure for the 8 Å radius system. Moreover, the weighted error in the interior potential was determined to be less than 0.37% and further reduced at higher element densities for both 4 Å and 8 Å charge density systems. The mean exterior potential is at most 0.004 kcal/mol, showing that the solution is physically accurate in both interior and exterior compared.

In contrast, the BEM result shows convergence to the exact/experimental values that are overall less accurate and slower with the increase in the element density. The weighted error for the interior potential is as high as 2%, and the exterior mean potential for the BEM solutions is as high as 0.29 kcal/mol for both 4 Å and 8 Å charge density systems, indicating incomplete cancellation of the field due to the fixed charges by the induced polarization density. The weaker convergence may be attributed to the relatively larger error in the total polarization charge associated with the BEM.

TABLE 1
Comparison between an embodiment of the present method (CON)
and BEM, with 4 Å Conducting Exterior Charge Radius

| | Solvation Energy | | Reaction Force | | Reaction Pressure | | | | Mean | |
|---|---|---|---|---|---|---|---|---|---|---|
| | (Kcal/mol) | | (Kcal/mol/Å) | | (Kcal/mol/Å) | | Interior potential | | Exterior Potential | |
| | (Exact = −19.75 | | (Exact = 1.88) | | (Exact = −1.88) | | % Error | | (Kcal/mol) | |
| # Elements | CON | BEM | CON | BEM | CON | BEM | CON | BEM | CON | BEM |
| 1280 | −19.85 | −20.22 | 1.91 | 1.96 | −1.90 | −1.99 | 0.37% | 2.1% | <0.004 | 0.29 |
| 2120 | −19.80 | −20.03 | 1.89 | 1.90 | −1.89 | −1.92 | 0.24% | 1.5% | <0.004 | 0.21 |
| 3440 | −19.79 | −19.99 | 1.88 | 1.88 | −1.90 | −1.90 | 0.24% | 1.3% | <0.004 | 0.19 |
| 5120 | −19.78 | −19.95 | 1.89 | 1.91 | −1.89 | −1.93 | 0.10% | 0.90% | <0.004 | 0.14 |
| 8288 | −19.76 | −19.88 | 1.88 | 1.89 | −1.88 | −1.90 | 0.06% | 0.66% | <0.004 | 0.10 |
| 13408 | −19.76 | −19.86 | 1.88 | 1.88 | −1.89 | −1.89 | 0.06% | 0.60% | <0.004 | 0.09 |

TABLE 2
Comparison between CON and BEM, with 8 Å Conducting Exterior Charge Radius.

| | Solvation Energy | | Reaction Force | | Reaction Pressure | | | | Mean | |
|---|---|---|---|---|---|---|---|---|---|---|
| | (Kcal/mol) | | (Kcal/mol/Å) | | (Kcal/mol/Å) | | Interior potential | | Exterior Potential | |
| | (Exact = −46.84) | | (Exact = 20.48) | | (Exact = −20.48) | | % Error | | (Kcal/mol) | |
| # Elements | CON | BEM | CON | BEM | CON | BEM | CON | BEM | CON | BEM |
| 1280 | −46.84 | −48.00 | 21.17 | 21.85 | −21.21 | −22.41 | 0.37% | 2.0% | <0.004 | 0.28 |
| 2120 | −46.29 | −46.83 | 20.68 | 20.97 | −20.77 | −21.22 | 0.15% | 1.0% | <0.004 | 0.15 |
| 3440 | −46.14 | −46.43 | 20.53 | 20.66 | −20.58 | −20.80 | 0.19% | 0.68% | <0.004 | 0.10 |
| 5120 | −46.29 | −46.81 | 20.68 | 20.97 | −20.77 | −21.23 | 0.10% | 0.89% | <0.004 | 0.13 |
| 8288 | −46.14 | −46.40 | 20.53 | 20.65 | −20.62 | −20.80 | 0.051% | 0.48% | <0.004 | 0.075 |
| 13408 | −46.10 | −46.25 | 20.50 | 20.56 | −20.50 | −20.63 | 0.022% | 0.32% | <0.004 | 0.051 |

Accordingly, the presently disclosed methods using continuum approximation provide a robust and fast computation of solvation effects on the molecule that is not only comparable but more accurate and precise than the conventional BEM approach. The presently disclosed methods may be utilized to simulate and calculate the binding affinity of molecules, such as drugs, hormones, and metabolites to proteins, and protein binding to other macromolecules such as other proteins and nucleic acids, where it is necessary to estimate the effects of solvent polarization. The method is robust to small perturbations in the surface geometry and provides convergent results that can be applied in numerous problem domains involving optimization and simulation of biomolecular systems.

FIGS. 7A through 7D illustrate example visual representations that the methods described in this document may generate and output on a display device. FIG. 7A illustrates a stick representation of a molecule 701 that the system may display. In this representation, the chemical bonds connecting atoms are visualized as thin tubes (sticks), and all atoms have an associated charge, typically a fractional value with magnitude less than one. The atoms themselves are not explicitly shown, their presence is implied at the termini of the sticks, or at their junctures. Hydrogen atoms with low atomic charge (such as non-polar hydrogens on alkyl chemical groups) are removed from the representation. In the representation, nitrogen atoms 702 are displayed in a first color or shade, while oxygen atoms 703 are represented in a second, darker color or shade. FIG. 7B illustrates a surface 705 that surrounds the molecule and that corresponds to a boundary of solvent contact with the molecule. Induced polarization charge on the surface is represented by shades, in this case regions of induced negative charge 707 are labeled “−”,while induced positive charge 708 is labeled “+”. Stronger density of shading indicates greater numerical magnitude.

The solvent will be attracted to the surface, and it will apply pressure to the atoms of the molecule at the locations the solvent is polarized. This interaction may be included in the digital representation that the system causes a display device to display. This is shown by way of example in FIG. 7C, which shows a visual representation of a molecule that includes graphic representations of the force vectors showing this addition, and where in this case vector 721 is represented as an arrow extending from the molecule in a direction and with a length that represents and corresponds to a force vector corresponding to through-space interaction between the induced polarization charge on the surface 705 and the corresponding charges of a localized group of the molecule's atoms. Vector 722 is represented as an arrow extending from the molecule in a direction and with a length that represents to the net effect of pressure on the molecule as experienced by the same group of atoms. The overall net solvent effect on the molecule at this location is illustrated by vector 723, which is the sum of vectors 721 and 722. This visualizes a simulation of the net solvent effect on the molecule.

Moreover, as each polarized atoms on the molecule are associated with a charge, the neighboring charged atoms may interact with each other in form of coulombic, or electrostatic interactions as illustrated by vector 724 in FIG. 7D's visual representation of the molecule. It is observed that the raw coulombic or electrostatic interaction (vector 724) between the charged atoms is countered by the solvent effect (vector 723), i.e., pointing in the opposite direction. When the two interactions are added, the total force, i.e., resultant of all the effects, is determined as vector 725. A visualization tool such as that shown in FIGS. 7A-7D can be integrated into another simulation tool, or it can be operated as a stand-alone simulation.

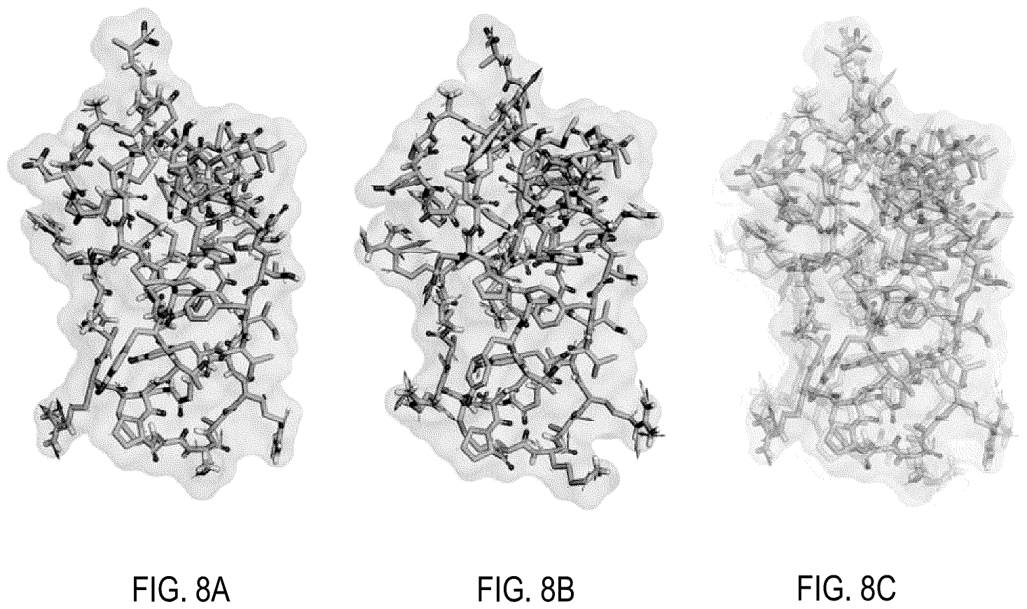

The mechanical forces due to solvent polarization computed using the method described here can be readily integrated with existing tools to expand their capabilities. FIGS. 8A-8C illustrate a dynamical simulation of a protein computed using GROMACS (GROningen MAchine for Chemical Simulation), another tool that may be used in the present method for molecular modeling and simulation. Here, the non-solvation mechanical forces (bond stretching, steric interactions between atoms, etc.) are computed using existing energy functions implemented in GROMACS, and the forces due to solvent polarization are computed externally by the methods described herein and incorporated into the simulation using constraint functions available in GROMACS. FIG. 8A shows the initial state at time 0 picoseconds, FIG. 8B after 10 picoseconds of simulation using the stochastic Langevin approach at temperature 300 K. The enclosing surface is re-generated and the forces due to solvent polarization are updated whenever any atom moves more than a specified threshold distance from its position at the prior update; here that threshold is set at 2.5 Å, equivalent to a typical atomic diameter. FIG. 8C shows these views overlaid to make the adaptation of the surface and computed forces over time more apparent. Various approaches are available to introduce solvation forces into GROMACS and other tools, and can be used to augment other capabilities of these tools, such as energy minimization and search for optimal conformations of molecules and configurations of interacting molecules.

The effects of solvent can be introduced by computing various components of the interaction energy associated with binding, principally: i) the change in solvation energy for a small biomolecule when binding to a protein, which is always a positive energy penalty; ii) the electrostatic interaction energy between the biomolecule and the protein, which consists of the sum of a direct coulombic contribution, plus the reaction field of the polarized solvent, which acts to screen the direct coulomb interaction. Existing methods for predicting affinities of drug-like molecules to bind to protein receptors do not include this detailed representation of solvent effects, but this capability can be immediately added using the methods described here.

### Example 2

Calculation of docking relationship between a collection of small molecules with the binding site of the kinase protein ABL2.

Another tool that may be utilized in the present method for predicting binding affinities of drug-like molecules is the program AUTODOCK VINA, which finds a best complementary fit, a process called “docking”, between small drug-like chemical compounds and a target protein receptor, producing a predicted binding position or “pose” for each small molecule. In this method, the molecules are ranked based on these poses using a phenomenological scoring function (the AUTODOCK score) that omits detailed physical interactions, but which has been trained to successfully identify molecules known to bind tightly to proteins.

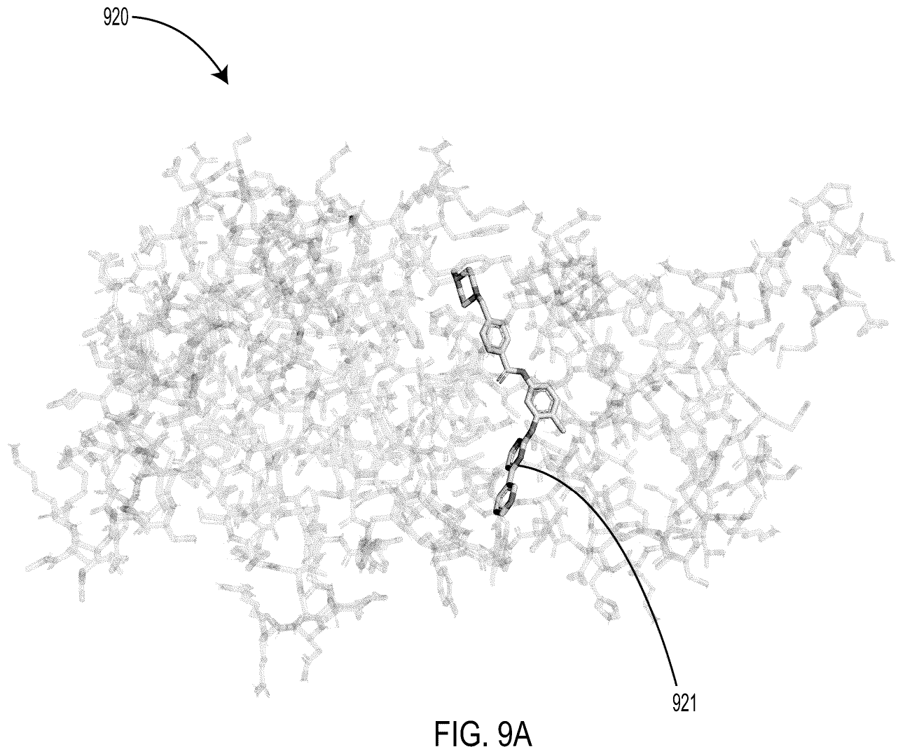

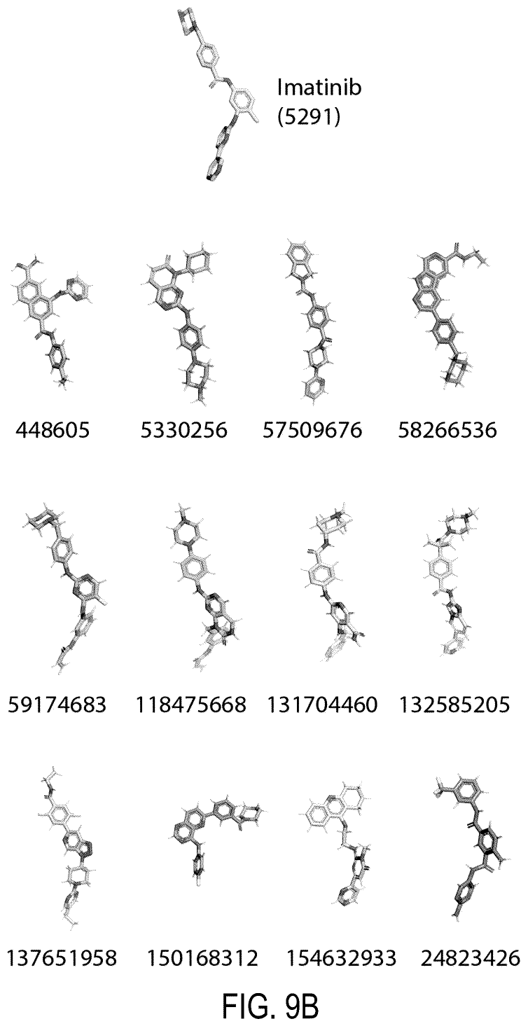

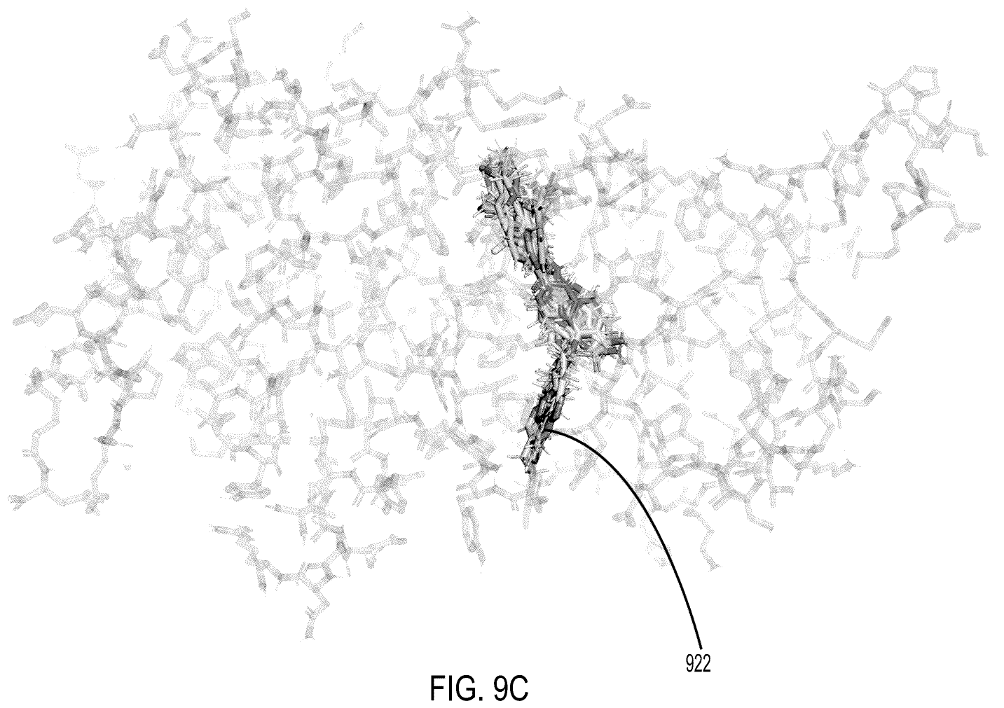

FIGS. 9A-9C show results for docking a collection of small molecules taken from the PubChem chemical database into the binding site of the kinase protein ABL2. FIG. 9A illustrates the experimental structure for the protein, molecule 920 (shown in partially transparent stick rendering), in complex with the compound Imatinib 921, a cancer therapeutic which is known to bind tightly to kinase proteins, in particular the important target ABL2. FIG. 9B shows Imatinib and an additional twelve compounds selected at random from the PubChem chemical library, based on similarity of structure and chemical properties to Imatinib. FIG. 9B shows the individual molecules labeled by their compound IDs (CIDs). FIG. 9C shows binding poses of Imatinib and the library molecules against the three-dimensional structure of the protein ABL2 as predicted using AUTODOCK VINA (thirteen total molecules from FIG. 9B, 922).

The solvation energy for each molecule in unbound form was computed by considering each molecule in isolation, in the same conformation as found by the docking prediction. The posed small molecules were then merged with the protein (as illustrated in FIG. 9C) and used to generate an overall surface that was unchanged throughout further computations; an initial solution of the induced surface charge density σ^{prot} was found for just the protein atoms. As described above, given that initial solution it was possible to then rapidly compute the individual distributions σ^{CID,prot} for each compound (identified by a unique PubChem identifier or CID), and using the same surface for the merged complex.

The solvation self-energy of each compound in the protein environment is then found as:

$${E}_{CID}^{s o l v} = {\sum }_{j ∈ CID} {\sum }_{k ∈ {S}^{prot}} \frac{{q}_{j}^{CID} {A}_{j} {σ}_{k}^{CID , prot}}{\vert {\vec{R}}_{j}^{CID} - {\vec{C}}_{k} \vert} , \qquad ( 18 )$$

where the atoms of the docked molecule have coordinates R^{CID}ⱼ and charges q^{CID}ⱼ, and the elements of the surface S^{prot} have centers Cₖ and areas Aₖ. This solvation energy is always less-stabilizing compared to the unbound state, as there is a loss of solvent exposure upon forming the complex, and represents a penalty (positive energy change) upon binding.

The screened interaction between the small molecule and the protein charges is found as:

$${E}_{inter}^{CIDprot} = {\sum }_{j ∈ CID} {\sum }_{k ∈ prot} \frac{{q}_{j} {q}_{k}}{\vert {\vec{R}}_{j}^{CID} - {\vec{R}}_{k}^{rot} \vert} + {\sum }_{j ∈ CID} {\sum }_{k ∈ {S}^{prot}} \frac{{q}_{j} {A}_{k} {σ}_{k}^{prot}}{\vert {\vec{R}}_{j}^{CID} - {\vec{C}}_{k} \vert} , \qquad ( 19 )$$

where the first term is the direct Coulomb interaction of the fixed atomic charges of the small molecule CID with the charges of the protein prot, and the second term represents the screening of these interactions due to the polarization of solvent by the protein charges, which is distributed on the protein surface S^{prot}.

Table 3 summarizes the detailed solvation results using the present method, and compares with the ranking score produced by AUTODOCK VINA. In all cases, energies/scores that are more negative represent stronger binding, while positive scores are inconsistent with the bound state. The known active compound, Imatinib, is verified on the basis of detailed electrostatics and solvation as having the lowest (best) energy of binding, and remarkably the computed energy matches the AUTODOCK score. Moreover, there is no correlation between detailed solvation energies and AUTODOCK scores for the other compounds, suggesting that the phenomenological AUTODOCK score is ineffective in ranking compounds that may be dissimilar to the compounds used to develop the original scoring function. While binding affinity for all of the selected compounds against ABL2 is not immediately available, none of the compounds except Imatinib is a known strong binder to this particular protein.

TABLE 3
Solvation energy of various small molecules docked to the binding site of the kinase protein ABL2 All values are in kcal/mol.

| | | | | | | | G | |
|---|---|---|---|---|---|---|---|---|
| | | | | D | E | | Predicted | |
| | A | | | Direct | Solvent | | Binding | |
| | Compound | B | C | Coulomb | Screening: | F | Energy, | |
| | Solvation: | Compound | Solvation | Interaction: | Compound − | Screening | Detailed | H |
| | Energy | Solvation: | Penalty | Compound − | Protein | Coulomb | Solvation | AUTODOCK |
| Compound ID (CID) | Unbound | Bound | (B − A) | Protein | Interaction | (D + E) | (C + F) | SCORE |
| 5291/IMATINIB | −78.8 | −32.80 | 46.00 | −168.60 | 110.10 | −58.50 | −12.50 | −12.6 |
| 24823426 | −11.80 | −0.10 | 11.70 | −14.20 | −0.50 | −14.70 | −3.00 | −11.3 |
| 59174683 | −14.00 | −0.10 | 13.90 | 8.10 | 0.10 | 8.30 | 22.10 | −11.2 |
| 57509676 | −11.60 | −0.20 | 11.50 | −6.10 | 2.70 | −3.40 | 8.10 | −11 |
| 131704460 | −79.10 | −29.70 | 49.30 | −173.70 | 122.60 | −51.10 | −1.80 | −10.3 |
| 132585205 | −82.00 | −58.70 | 23.30 | −130.40 | 124.50 | −5.90 | 17.40 | −10.2 |
| 154632933 | −10.40 | 0.00 | 10.30 | −0.40 | 0.30 | −0.10 | 10.30 | −9.9 |
| 58266536 | −72.30 | −15.20 | 57.00 | −150.70 | 130.90 | −19.80 | 37.30 | −9.8 |
| 448605 | −100.3 | −13.90 | 86.40 | −145.20 | 120.60 | −24.60 | 61.80 | −9.7 |
| 150168312 | −86.70 | −71.40 | 15.30 | −143.30 | 134.20 | −9.20 | 6.10 | −9.6 |
| 137651958 | −14.50 | −0.20 | 14.30 | −1.10 | 1.20 | 0.10 | 14.40 | −9.3 |
| 118475668 | −78.5 | −27.8 | 50.7 | −146 | 106.2 | −39.8 | 10.9 | −9.2 |
| 5330256 | −73.30 | −15.40 | 57.90 | −135.90 | 129.60 | −6.30 | 51.60 | −9.1 |

### Example 3

An important application of continuum solvation methods as described here is the prediction of the ionization states for amino acids in protein structures. The amino acids at the ends (N- and C-termini) of a protein polypeptide chain have terminal chemical groups that can be ionized (+1 at N-terminus, −1 at C-terminus), and the side chains of several amino acids can independently ionize, with +1 charge possible for Lysine, Arginine and Histidine, and −1 for Aspartate and Glutamate. Free amino acids (unconnected in a polypeptide chain) have all available sites ionized, while in a folded protein structure the local environment may feature reduced solvent exposure for some amino acids compared to the free state, and moreover there are electrostatic interactions with other amino acid sidechains, and also with the polypeptide backbone. These interactions may be more or less screened by the polarization of solvent, dependent upon the solvent exposure and specific geometry of the interacting amino acids.

The tendency of a chemical group to ionize is measured by the pK_{α}, the negative log₁₀ of the dissociation constant K_{α} for deprotonation of the group. The pK_{α} values have been determined by experiment for free amino acids and in short polypeptides; by applying our continuum electrostatic methods as described here, the shift in energy for ionization for a chemical group can be computed in an isolated reference compound, and compared against the energy computed in a distinct environment, such as a protein structure. The shift in energy upon ionization for protein group K (which can represent an amino acid side-chain or polypeptide chain terminus) can be expressed as the sum of shifts in solvation for the group and interaction with the rest of the protein. In one embodiment this may be implemented by defining the changes Δqₖ^{K,ion} in the atomic charges k of group K that move it from neutral to ionized state, and the change in the protein induced surface charge distribution in response to this change, symbolized Δσ^{K,ion}. The change in energy of the neutral system is then found as:

$$Δ {E}_{K}^{ion} = \frac{1}{2} {\sum }_{k ∈ K} {\sum }_{s ∈ {S}^{prot}} \frac{Δ {q}_{k}^{K , ion} Δ {σ}_{s}^{K , ion} {A}_{s}}{\vert {\vec{R}}_{k}^{K} - {\vec{C}}_{s} \vert} + {\sum }_{k ∈ K j} {\sum }_{∈ J \neq K} \frac{Δ {q}_{k}^{K , ion} {q}_{j}^{J , n e u t}}{\vert {\vec{R}}_{k}^{K} - {\vec{R}}_{j}^{J} \vert} + {\sum }_{k ∈ K} {\sum }_{{s ∈ {S}^{prot}}_{}} \frac{Δ {q}_{k}^{K , ion} {σ}_{s}^{n e u t} {A}_{s}}{\vert {\vec{R}}_{k}^{K} - {\vec{C}}_{s} \vert} . \qquad ( 20 )$$

In equation (20), the first term is the solvation energy for ionizing group K, the second is the energy change due to the Coulomb interaction with the rest of the protein groups (taken in neutral form), and the last term is screening due to solvation of this Coulomb interaction. It is assumed throughout an unchanging protein surface S^{prot} with element centroids Cₛ and areas Aₛ. (It is noted that this expression includes a Coulombic “self-interaction” of group K with itself, which is approximately constant for a given chemical group, and may be positive depending on the charge model used.)

The change in pK_{α} for group K can now be computed using a well-known relationship:

$$Δ p {K}_{a}^{K} = \pm \frac{1}{2.303 R T} ( Δ {E}_{K}^{ion} - Δ {E}_{reference ( K )}^{ion} ) . \qquad ( 21 )$$

In equation (21), the “reference” contribution is the energy change computed for an appropriate model of the chemical group K, typically featuring the same chemical structure but in a small, fully solvated compound. The +/− sign indicates that the energy change that corresponds to deprotonation of group K must be considered, which means that the sign of the energy change in this formula must be inverted if ionization implies adding a proton (the case for basic groups, e.g. Arginine amino acid sidechains). In equation (21), R is the gas constant and T the temperature.

The preceding presentation assumes only one group being ionized in a neutral protein. This may be extended to compute pairwise interactions, where site K ionizes with site J already ionized:

$$Δ {E}_{K , J}^{inter} = {\sum }_{k ∈ K} {\sum }_{s ∈ {S}^{prot}} \frac{Δ {q}_{k}^{K , ion} {Δσ}_{s}^{J , ion} {A}_{s}}{\vert {\vec{R}}_{k}^{K} - {\vec{C}}_{s} \vert} + {\sum }_{j ∈ J} {\sum }_{s ∈ {S}^{prot}} \frac{Δ {q}_{k}^{J , ion} Δ {σ}_{s}^{K , ion} {A}_{s}}{\vert {\vec{R}}_{j}^{J} - {\vec{C}}_{s} \vert} . \qquad ( 22 )$$

The total pK_{α} shift for residue K is then:

$$Δ p {K}_{a}^{K} = \pm \frac{1}{2.303 R T} ( Δ {E}_{K}^{ion} + {\sum }_{J ∈ \{ Ionized \} , J \neq K} Δ {E}_{K , J}^{inter} - Δ {E}_{reference ( K )}^{ion} ) . \qquad ( 23 )$$

where the interaction sum is over the set of all ionized residues {Ionized} corresponding to an assumed configuration of charged states.

In some embodiments it may be found necessary to consider many such combinations of charge states, and evaluate their individual energies and probabilities, but for the present discussion, only interactions of large magnitude, noting that most group interactions are found to be very small (<0.1 kcal/mol in magnitude) is considered. Thus, it is adequate in most cases to consider only the “intrinsic” energy change for ionizing a single residue.

Again, it is stressed that the methods described here support the rapid computation of solvation effects for multiple arrangements of fixed charges. In the present case this means the computation of the polarization charge for the neutral state, followed by a rapid re-computation for all of the single-group ionization states. Shifts in pK_{α} for groups considered in isolation can then be computed using equation (21), with large inter-group contributions included for strong interactions using equation (23).

Table 4 presents result for application of this approach to estimating the ionization states of amino acid sidechains and also the peptide termini for the enzyme Lysozyme, using the same structure illustrated in FIGS. 5A and 5B. The table columns in order list: (A) the identity of the ionizable group, using standard abbreviations for the amino acids and their numerical position in the polypeptide sequence; (B) the charge of the group when ionized; (C) the ionization energy computed using the detailed solvation methodology just presented; (D) the ionization energy of a reference structure appropriate for the group; (E) the difference in energy with respect to the reference (i.e., =D−E); (F) identity of strong interaction if present; (G) energy of strong interaction if present; (H) total energy shift (=E+G); unshifted pK_{α} of group; (J) pK_{α} shift computed from H; (K) shifted pK_{α} (I+J); (L) predicted ionization at neutral pH=7. It should be understood the physically-meaningful number is the difference between the value found in the protein versus that found in a well-solvated reference structure. A negative difference corresponds to greater stabilization of the ionized state in the protein environment, while a positive difference means a lower probability of ionization.

TABLE 4
Ionization states of amino acid sidechains and peptide termini for the Lysozyme. All energy values are in kcal/mol.

| | | | | | F | | H | | | | |
|---|---|---|---|---|---|---|---|---|---|---|---|
| | B | | D | | Interactive | | Total | | | | |
| A | Charge | C | Reference | E | Residue | G | Ionization | I | J | K | L |
| Ionizable | State | Ionization | Ionization | Energy | (magnitude > | Interaction | Energy | Unshifted | pKA | Final | Predicted |
| Group | (Ionized) | Energy | Energy | Shift | 1 kcal/mol) | Energy | Shift | pKA | Shift | pKA | Charge |
| N-terminus | 1 | 82.8 | 94.1 | −11.3 | Lysine−1 | 1.8 | −9.5 | 9.0 | 6.9 | 15.9 | 1 |
| (Lysine-1) | | | | | (sidechain) | | | | | | |
| Lysine-1 | 1 | 83.5 | 81.9 | 1.6 | N−terminus | 1.8 | 3.4 | 10.5 | −2.5 | 8.0 | 1* |
| Arginine-5 | 1 | −226.5 | −228 | 1.5 | | | 1.5 | 12.5 | −1.1 | 11.4 | 1 |
| Glutamate-7 | −1 | −129.1 | −128.1 | −1.0 | | | −1 | 4.3 | −0.7 | 3.6 | −1 |
| Lysine-13 | 1 | 83.1 | 81.9 | 1.2 | | | 1.2 | 10.5 | −0.9 | 9.6 | 1 |
| Arginine-14 | 1 | −227.7 | −228 | 0.3 | | | 0.3 | 12.5 | −0.2 | 12.3 | 1 |
| Histidine-15 | 1 | −95.8 | −120 | 24.2 | | | 24.2 | 6.0 | −17.6 | −11.6 | 0 |
| Aspartate-18 | −1 | −132 | −127.4 | −4.6 | | | −4.6 | 3.7 | −3.4 | 0.3 | −1 |
| Arginine-21 | 1 | −224.4 | −228 | 3.6 | | | 3.6 | 12.5 | −2.6 | 9.9 | 1 |
| Lysine-33 | 1 | 82.7 | 81.9 | 0.8 | | | 0.8 | 10.5 | −0.6 | 9.9 | 1 |
| Glutamate-35 | −1 | −125.7 | −128.1 | 2.4 | | | 2.4 | 4.3 | 1.7 | 6.0 | −1* |
| Arginine-45 | 1 | −213.5 | −228 | 14.5 | Aspartate−66 | −4.1 | 10.4 | 12.5 | −7.6 | 4.9 | 0 |
| Aspartate-48 | −1 | −102.6 | −127.4 | 24.8 | Arginine−61 | −32.5 | −7.7 | 3.7 | −5.6 | −1.9 | −1 |
| Aspartate-52 | −1 | −127.3 | −127.4 | 0.1 | | | 0.1 | 3.7 | 0.1 | 3.8 | −1 |
| Arginine-61 | 1 | −205.6 | −228 | 22.4 | Aspartate−48 | −32.5 | −10.1 | 12.5 | 7.4 | 19.9 | 1 |
| Aspartate-66 | −1 | −142.1 | −127.4 | −14.7 | Arginine−45 | −4.1 | −18.8 | 3.7 | −13.7 | −10.0 | −1 |
| Arginine-68 | 1 | −226 | −228 | 2.0 | | | 2 | 12.5 | −1.5 | 11.0 | 1 |
| Arginine-73 | 1 | −223.2 | −228 | 4.8 | | | 4.8 | 12.5 | −3.5 | 9.0 | 1 |
| Aspartate-87 | −1 | −129.3 | −127.4 | −1.9 | | | −1.9 | 3.7 | −1.4 | 2.3 | −1 |
| Lysine-96 | 1 | 85.7 | 81.9 | 3.8 | | | 3.8 | 10.5 | −2.8 | 7.7 | 1* |
| Lysine-97 | 1 | 78.1 | 81.9 | −3.8 | | | −3.8 | 10.5 | 2.8 | 13.3 | 1 |
| Aspartate-101 | −1 | −132 | −127.4 | −4.6 | | | −4.6 | 3.7 | −3.4 | 0.3 | −1 |
| Arginine-112 | 1 | −226.1 | −228 | 1.9 | | | 1.9 | 12.5 | −1.4 | 11.1 | 1 |
| Arginine-114 | 1 | −22 | −228 | 6.0 | | | 6 | 12.5 | −4.4 | 8.1 | 1* |
| Aspartate-119 | −1 | −131.5 | −127.4 | −4.1 | | | −4.1 | 3.7 | −3.0 | 0.7 | −1 |
| Arginine-128 | 1 | −227.7 | −228 | 0.3 | | | 0.3 | 12.5 | −0.2 | 12.3 | 1 |
| C-terminus | −1 | −118.9 | −119.4 | 0.5 | | | 0.5 | 2.4 | 0.4 | 2.8 | −1 |
| (Leucine-129) | | | | | | | | | | | |

*Predicted as weakly ionized

While most of the residues are predicted to be in ionized state, Histidine-15 is largely buried and is poorly-stabilized by solvent, leading to predicted neutral state, which is in agreement with experiment. Some of the residues are classified as “weakly ionized”, as indicated by “*” meaning that their predicted pKa is close to the numerical value of neutral solution pH (7.0), and so are expected to be ionized only part of the time. Remarkably, one of these, Glutamate-35 has a predicted pKa of 6.0, close to experimental estimates of 6.3. This amino acid sidechain has been carefully studied, as it is widely believed to be kept in a weakly ionized state by the protein environment, supporting its functional role in the enzymatic action of this protein.

### Example 4

It is important to compute the electric field in the exterior of a protein or other macromolecule to support many applications, such as estimating the distribution of salt ions in the vicinity of the molecule, or to compute long-range electrostatic interactions between molecules. The methods disclosed herein directly compute the surface polarization charge corresponding to the case of conducting exterior and so set the exterior potential to exactly zero. However, by using a simple approximation, the conducting solution for the polarization charge density can be related to the perturbation δσ, the difference between the polarization density for non-conducting exterior versus the conducting case:

$$δ σ ( \vec{R} ) = {σ}^{noncond} ( \vec{R} ) - {σ}^{cond} ( \vec{R} ) = - \frac{{ϵ}_{i}}{{ϵ}_{o}} {σ}^{cond} ( \vec{R} ) . \qquad ( 24 )$$

In equation (24), R is the position on the molecular surface, and εᵢ and εₒ are respectively the dielectric constants for the interior and exterior regions, as defined by the surface. The interior dielectric constant for macromolecules such as proteins is typically taken in the range between 1-4, the exterior dielectric constant corresponding to aqueous solvent is taken in the range 78-80. This “perturbation” approximation is actually exact for spherical surfaces with fixed charge at center, and should remain a good approximation whenever atoms are physically enclosed by partial spherical surfaces, which is the case for molecular solvent-accessible surfaces as used here.

Since the conducting solution for the polarization charge nullifies the potential of the atomic fixed charges in the exterior, the exterior potential is computed using only the perturbation distribution δσ:

$${Φ}_{e x t} ( \vec{p} ) = {\sum }_{s ∈ S} \frac{δ {σ}_{s} {A}_{s}}{\vert \vec{p} - {\vec{C}}_{s} \vert} . \qquad ( 25 )$$

In equation (25) the exterior potential is computed at position p, and arises from the perturbation distribution computed using equation (24), with charge densities defined on surface elements with areas Aₛ and center positions Cₛ.

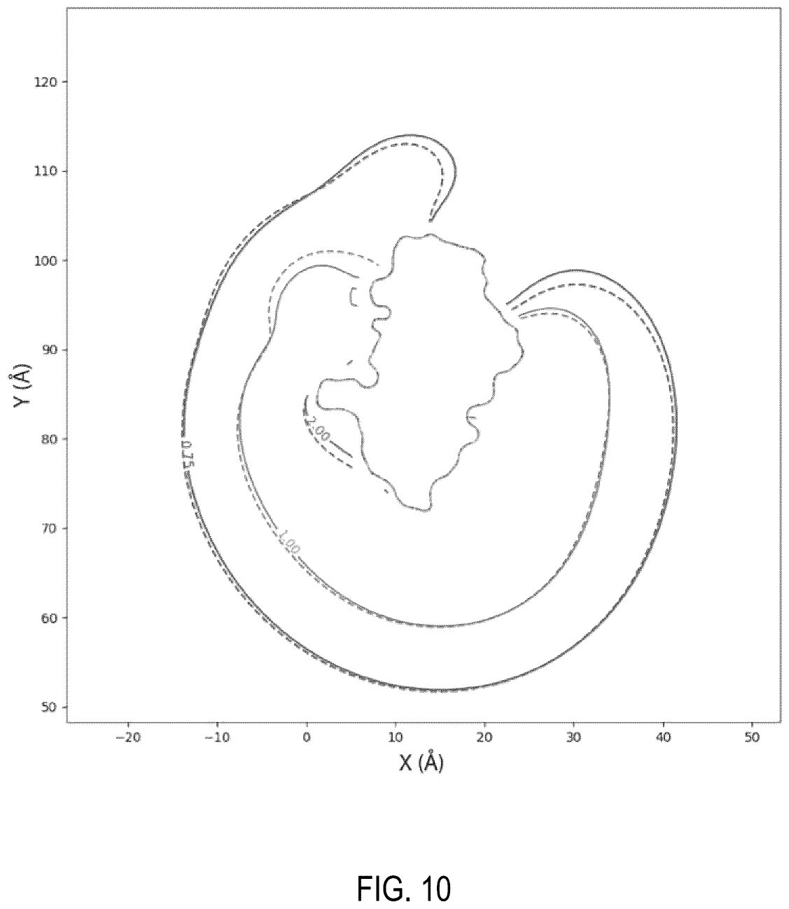

As discussed previously, the boundary element method (BEM) is capable of rigorously computing the distribution δσ, albeit with higher computational cost than the methods disclosed herein. A computation was carried out for the protein Bovine Pancreatic Trypsin Inhibitor (BPTI) using the methods here disclosed and the alternative BEM, and the electric potential computed over the points of a regular grid using the results of both methods. The weighted difference between the alternate solutions for the exterior potential is found to be 7%, validating the economical approximation of equation (24). FIG. 10 shows a cross-section through the protein, and iso-contours of electric potential found using the methods disclosed here (solid lines) and the BEM (dashed lines), showing good agreement between the approximation equation (24) and the physically rigorous results of the BEM.

### Example 5

An important application of continuum electrostatics is to compute energies and forces in periodic systems that model an infinite membrane structure. This is of high interest given that most drug targets are in fact transmembrane receptor proteins that span the cell membrane from the cell exterior to its cytoplasmic interior, and to realistically model these requires a representation that includes the protein and portion of the surrounding lipid membrane. In dynamical simulations with explicit solvent, the simulation is typically carried out with periodicity in two dimensions, effectively generating an infinite system from the protein and a rectangular region of surrounding membrane.

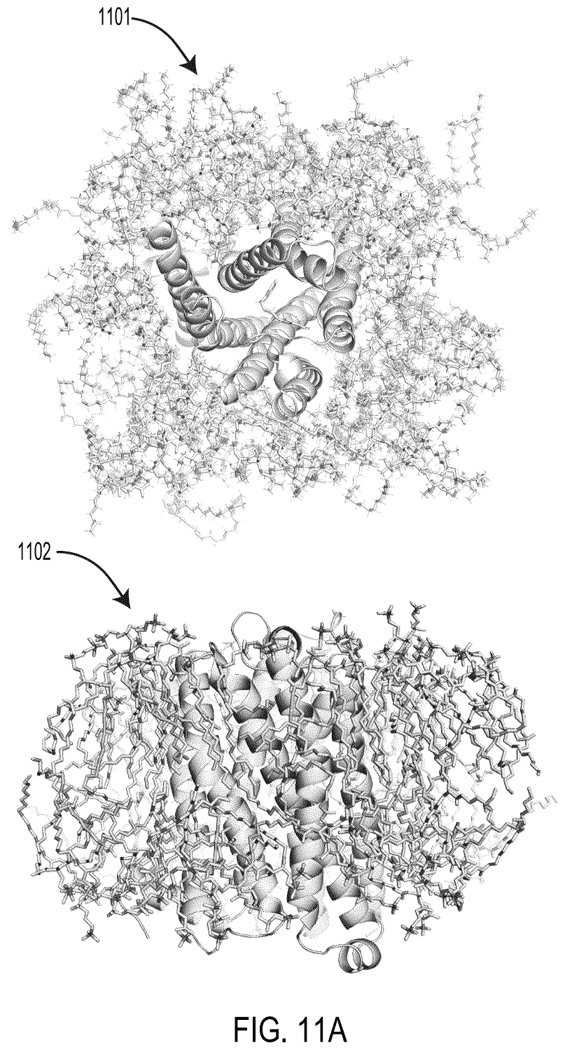

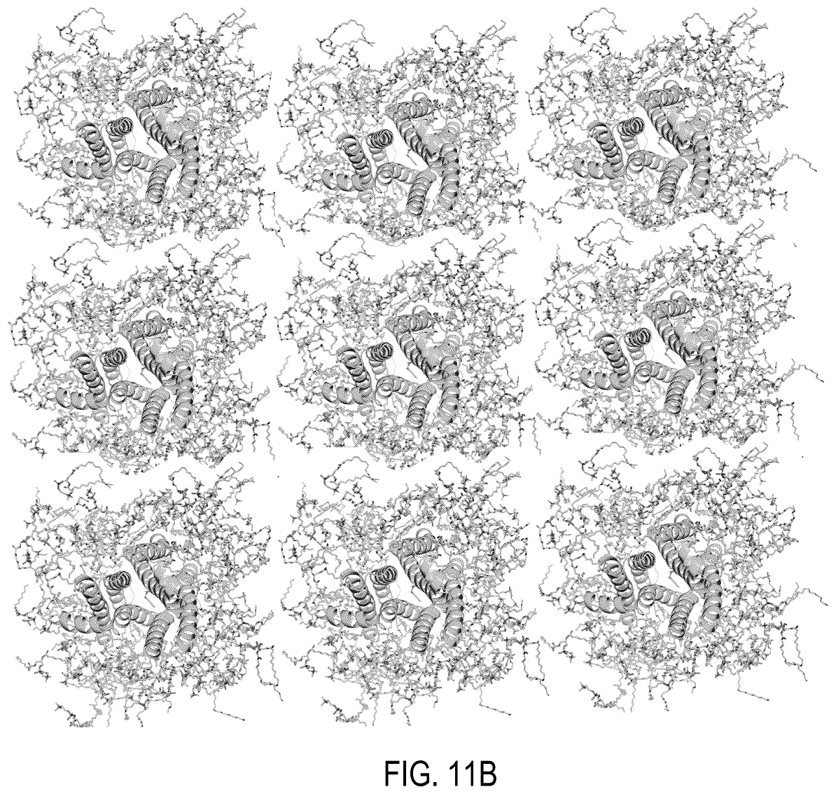

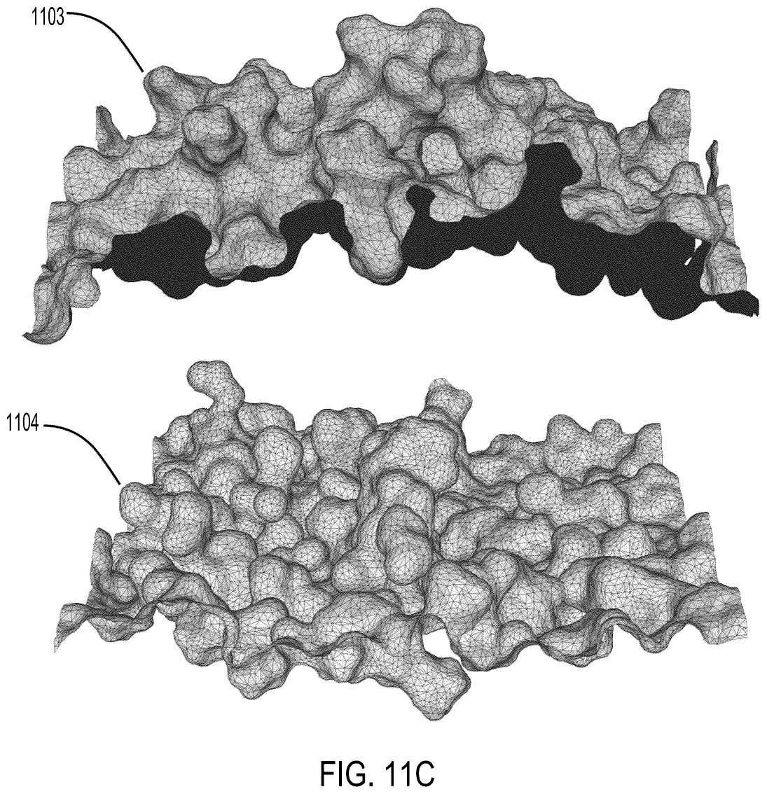

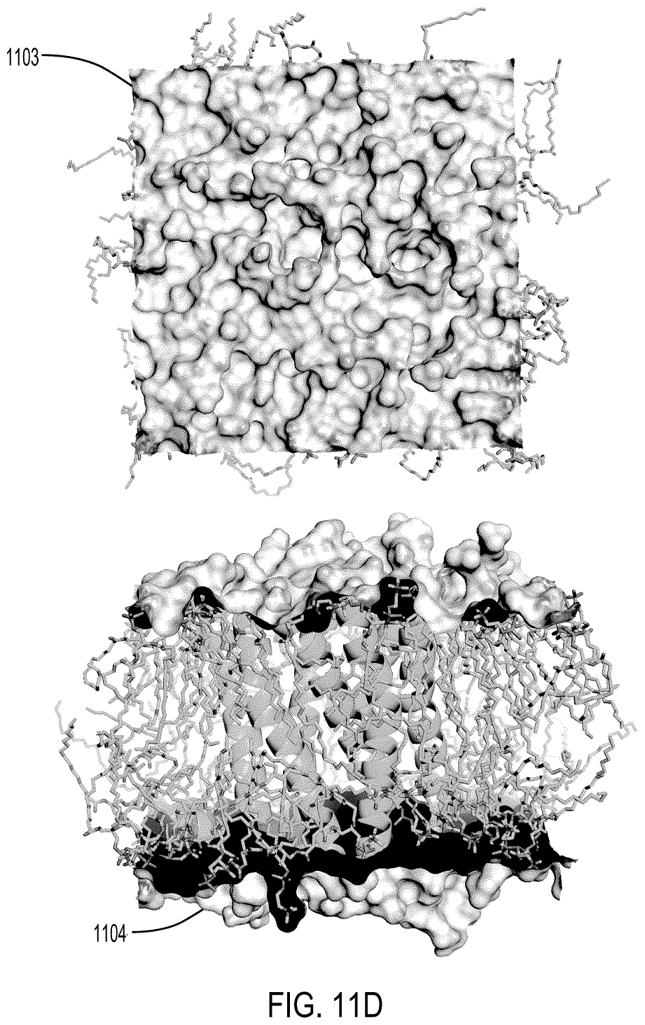

FIG. 11A shows a unit cell for a membrane system that includes the human Serotonin-2A receptor (shown as cartoon rendering) packed with lipid molecules (shown in tube rendering). 1101 is a view from the extracellular side of the membrane, 1102 is a view perpendicular to the plane of the membrane. This unit cell can be tiled as suggested in FIG. 11B to implicitly model an infinite membrane structure. A molecular solvent-accessible surface can be generated for the single central periodic cell by including all the atoms of the central cell and a portion of neighboring periodic cells, then generating surface using probe positions for the central cell only. FIG. 11C. shows the extracellular membrane elements 1103 and intracellular membrane elements 1104 generated for the membrane system; here the intra-cellular surface elements are viewed from the rear.

Field points may be generated for the membrane surface in exactly the same way as disclosed above for other molecular systems, the distribution of induced polarization charge can be computed which sets the electric potential at the field points to zero. FIG. 11D shows the computed distribution of polarization charge arising in response to the atomic charges in the central periodic cell, for both the extracellular membrane elements 1103 and intracellular 1104. In this illustration the intensity of induced polarization charge is indicated by darkness of shading, but polarity is not indicated.

In various embodiments, this initial estimate of the polarization charge may be found adequate, but the distribution may contain isolated regions of high magnitude near the edges of the membrane, which is due to the approximation of modeling an implicitly infinite periodic system using only the single central cell.

To address this discrepancy, initial estimate of the polarization charge error over the field points is set approximately to zero:

$$\vec{e r r} = K {\vec{σ}}^{i n i t i a l} + {E}_{Q , cell 0 , 0}^{F} ≅ 0. \qquad ( 26 )$$

However the potential at the field points involves only the central cell and ignores the rest of the periodic system; this is highlighted in equation (26), which is identical in form to equation (10), but with potential defined as the fixed charges as E^{F}_{Q,cell0,0} to stress that the initial estimate of induced polarization charge σ^{initial} only nullifies the potential at the field points due to the field of the fixed charges of the central periodic cell.

Accordingly, the error is updated to include the contributions of all images of the central cell by taking lattice sums over both the fixed charges and the surface elements, which all carry countercharge corresponding to σ^{initial}. Assuming the membrane cells lie in the plane perpendicular to the z-axis, with periodic translations ΔX and ΔY in the x- and y-directions, the contribution at the field points due to the fixed charges outside the central cell is:

$${E}_{Q ( lattice )}^{F , α} = {\sum }_{- ∞ < M < ∞} {\sum }_{- ∞ < N < ∞} {\sum }_{j ∈ Q} \frac{{q}_{j}}{\vert {\vec{R}}_{F}^{α} - ( {\vec{R}}_{Q}^{j} + M \vec{Δ X} + N \vec{Δ Y} ) \vert} , \qquad ( 27 )$$

where equation (27) expresses the infinite sum of the electric potential at field point α due to all displacements of the charges of the central cell over the lattice, and which will be evaluated using existing well-known and fast methods (based on application of the Fourier transform) for evaluating such sums.

Similarly, the potential due to the initial estimate of the induced polarization charge is found as:

$${E}_{{σ}^{initial} ( lattice )}^{F , α} = {\sum }_{- ∞ < M < ∞} {\sum }_{- ∞ < N < ∞} {\sum }_{S ∈ s} \frac{{σ}_{s}^{i n i t i a l} {A}_{s}}{\vert {\vec{R}}_{F}^{α} - ( {\vec{C}}_{s} + M \vec{Δ X} + N \vec{Δ Y} ) \vert} , \qquad ( 28 )$$

where the sum involves the positions and areas of all elements of the membrane surface for the central cell. (In the sums in equations (27) and (28), it is assumed that M and N are not zero, as the contribution of the central cell to the potentials has already been explicitly evaluated.)

Equation (26) may be recast to include the error due to the periodic system, and introduce an update Δσ^{lattice} to the induced polarization charge to force the potential at the field points to zero:

$$\vec{err} = ( K {\vec{σ}}^{i n i t i a l} + {E}_{Q , cell 00}^{F} ) + ( {E}_{Q ( lattice )}^{F} + {E}_{{σ}^{initial} ( lattice )}^{F} ) + K \vec{Δ} {σ}^{lattice} = 0. \qquad ( 29 )$$

Equation (29) can be solved for Δσ^{lattice} using exactly the same methods already disclosed, with the four terms in parentheses forming an updated total error.

Given a solution for Δσ^{lattice}, the solution then can be updated for the induced polarization charge:

$${\vec{σ}}^{update} = {\vec{σ}}^{initial} + {\vec{Δσ}}^{lattice} . \qquad ( 30 )$$

There will still be some residual error even after introduction of the updated polarization density, as the updated density depends on the lattice sum (equation (28)), which in turn depends on the current estimate of the polarization charge. To further improve accuracy, the algorithm may be iterated as many times as desired, with σ^{initial} replaced in each iteration by the latest update, σ^{update}.

Using the methods just described, all of the approaches disclosed above for dynamical simulation, docking of small molecules, identification of ionization states, and any other application that requires computation of solvation energies and forces, may be immediately extended to handle periodic infinite membrane models.

Moreover, the same approach applies directly to any system constructed by symmetry operations, such as the structure of the protein coat of a virus, which is generated by applying symmetry operations to a single protein motif. In the more general case, the infinite lattice sums of equations (27) and (28) are replaced by finite sums over the symmetry operations needed to generate the entire system from a starting motif.

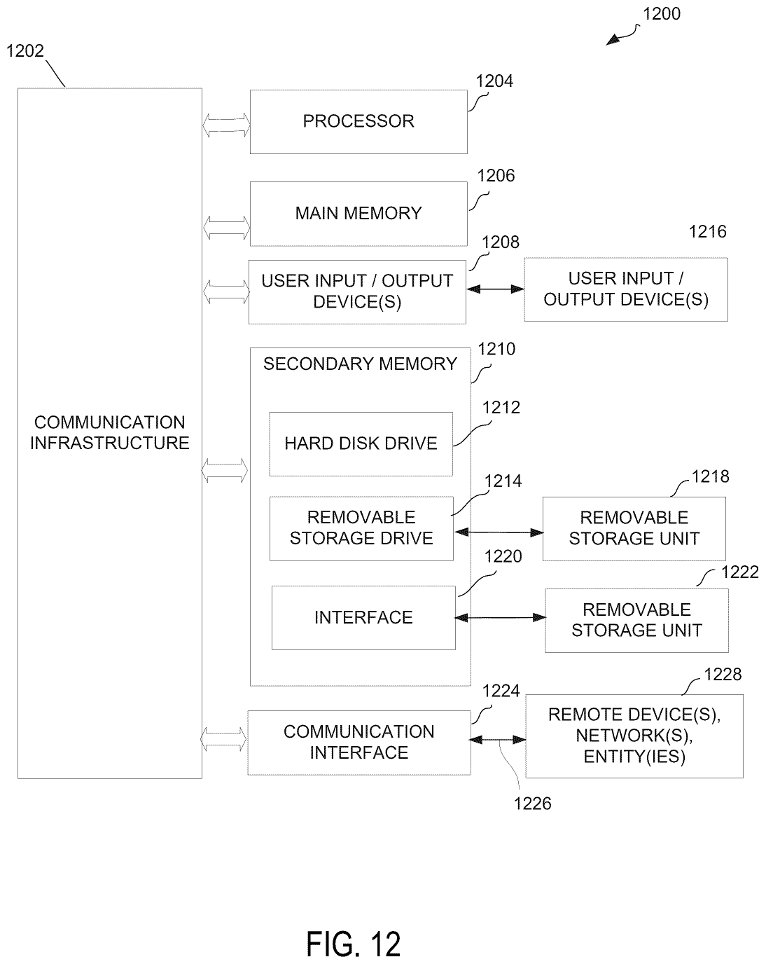

The methods described in this document may be implemented by a computing device, or by a computer program product comprising a memory and computer programming instructions. Elements of an example computing device and/or system 1200 are disclosed in FIG. 12. Computer system can be any computer capable of performing the functions described in this document.

Computer system 1200 includes one or more processors (also called central processing units, or CPUs), such as a processor 1204. Processor 1204 is connected to a communication infrastructure or bus 1202. Optionally, one or more of the processors 1204 may each be a graphics processing unit (GPU). In various embodiments, a GPU is a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 1200 also includes user input/output device(s) 1216, such as monitors, keyboards, pointing devices, etc., that communicate with communication infrastructure or bus 1202 through user input/output interface(s) 1208. In various embodiments, at least one of the input/output device(s) 1216 is a display monitor or other display device with a display screen.

Computer system 1200 also includes a main or primary memory 1206, such as random access memory (RAM). Main memory 1206 may include one or more levels of cache. Main memory 1306 has stored therein control logic (i.e., computer software) and/or data.

Computer system 1200 may also include one or more secondary memory devices 1210. Secondary memory device 1210 may include, for example, a hard disk drive 1212 and/or a removable storage device or drive 1214. Removable storage drive 1214 may be an external hard drive, a universal serial bus (USB) drive, a memory card such as a compact flash card or secure digital memory, a compact disc drive, an optical storage device, a tape backup device, and/or any other storage device/drive.

Removable storage drive 1214 may interact with a removable storage unit 1218. Removable storage unit 1218 includes a computer usable or readable storage device having stored thereon computer software (control logic) and/or data. Removable storage unit 1218 may be an external hard drive, a universal serial bus (USB) drive, a memory card such as a compact flash card or secure digital memory, a compact disc, an optical storage disk, and/any other computer data storage device. Removable storage drive 1214 reads from and/or writes to removable storage unit 1218 in a well-known manner.

According to an example embodiment, secondary memory device 1210 may include other means, instrumentalities or other approaches for allowing computer programs and/or other instructions and/or data to be accessed by computer system 1200. Such means, instrumentalities or other approaches may include, for example, a removable storage unit 1222 and an interface 1220. Examples of the removable storage unit 1222 and the interface 1220 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 1200 may further include a communication or network interface 1224. Communication interface 1224 enables computer system 1200 to communicate and interact with any combination of remote devices, remote networks, remote entities, etc. (individually and collectively referenced by reference number 1228). For example, communication interface 1224 may allow computer system 1200 to communicate with remote devices 1228 over communications path 1226, which may be wired and/or wireless, and which may include any combination of LANs, WANs, the Internet, etc. Control logic and/or data may be transmitted to and from computer system 1200 via communication path 1226.

In some embodiments, a tangible, non-transitory apparatus or article of manufacture comprising a tangible, non-transitory computer useable or readable medium having control logic (software) stored thereon may be referred to in this document as a “computer program product” or program storage device. This includes, but is not limited to, computer system 1200, main memory 1206, secondary memory device 1210, and removable storage units 1218 and 1222, as well as tangible articles of manufacture embodying any combination of the foregoing. Such control logic, when executed by one or more data processing devices (such as computer system 1200), causes such data processing devices to operate as described in this document.

The methods and systems described in this document have many practical applications. Understanding a solvent's effect on a molecule is very important in the development of pharmaceuticals and other chemical compounds and solutions. For example, understanding how a molecule will react to a particular solvent will be important if the molecule will be delivered in a solution or suspension. It will also be important to understand a molecule's absorption rate in the human body when ingested or otherwise taken into the body.

The methods describe above can be used to compute the energies resulting from an aqueous solvent in a model of a molecule. By computing the polarization charge distribution due to the atomic charges of the model, and by summing the through-space electrostatic energy contributions due to the interactions between the polarization charges on the elements and the charges on the atoms of the model, interaction between the molecule and the solvent can be better understood.

Based on the teachings contained in this disclosure, it will be apparent to persons skilled in the relevant art(s) how to make and use embodiments of this disclosure using data processing devices, computer systems and/or computer architectures other than that shown in FIG. 12.

The features and functions described above, as well as alternatives, may be combined into many other different systems or applications. Various alternatives, modifications, variations or improvements may be made by those skilled in the art, each of which is also intended to be encompassed by the disclosed embodiments.

1. A method for providing a digital computer simulation of effects of a solvent on a molecule, the method comprising, by a processor:
accessing a model of a molecule, the model comprising a digital representation of a collection of atoms each with radius and electric charge;
defining a surface that surrounds the molecule of the model and that corresponds to a boundary of solvent contact with the molecule, and partitioning the surface using discrete surface elements, wherein the surface elements provide a non-spherical surface on the molecule;
defining a plurality of field points in the solvent near the surface elements of the molecule;
using the model, the surface elements and the field points to compute a distribution of polarization charge induced on the surface by an electric field corresponding to interaction of the solvent with the electric field and consequent polarization of the solvent;
measuring one or more mechanical effects on the molecule due caused by the polarization charge and by pressure exerted by the polarization charge;
generating a visual representation of the distribution of the polarization charge induced on the surface by the electric field corresponding to the interaction of the solvent with the electric field and consequent polarization of the solvent; and
causing a display device to output the visual representation.

2. The method of claim 1, wherein partitioning the surface comprises defining the surface elements to include or neighbor one or more of the atoms of the molecule.

3. The method of claim 1, wherein measuring the one or more mechanical effects comprises measuring a direct through-space force at each of the atoms due to the electric field caused by the induced distribution of polarization charge acting upon a charge associated with the atom.

4. The method of claim 1, further comprising measuring a pressure exerted by polarization density defined on the surface elements.

5. The method of claim 1, wherein defining the field points comprises deriving the field points from one or more of the following:
spherical probes that are offset from and in contact with the surface;
a regular grid exterior to the surface; or
a combination of the spherical probes and the regular grid around the molecule.

6. The method of claim 5, wherein:
the field points comprise centers of the probes; and
defining the probes comprises positioning the probes normal to the surface elements at a separation equal to a solvent radius.

7. The method of claim 5, wherein:
the field points comprise centers of the probes; and
the probes are spherical and comprise a solvent radius of about 1.5 Å.

8. The method of claim 1, wherein measuring the pressure exerted by the polarization charge comprises, for each surface element, using a probe offset from the surface element to apply a pressure vector to the surface element, wherein the probe transfers contact forces to the atoms of the collection of atoms that the probe contacts.

9. The method of claim 1, further comprising measuring total pressure exerted by the polarization charge on the atoms, by adding individual contact forces exerted on individual atoms by a pressure of the surface elements.

10. The method of claim 1, wherein:
the field points comprise a grid; and
defining the grid comprises:
defining a series of voxelized volume elements exterior to the surface of the molecule;
providing grid points at centers of the voxelized volume elements; and
generating a layer of the grid points around the molecule using volume elements that adjoin the surface.

11. The method of claim 1, wherein the measuring one or more mechanical effects includes determining one or more properties of a molecule: solvation energy, reaction force, reaction pressure, coulomb interaction, binding energy, charge state, ionization energy, interaction energy, or pKa.

12. A computer program product comprising a memory device that stores programming instructions that are configured to cause a processor to provide a digital computer simulation of effects of a solvent on a molecule by:
accessing a model of a molecule, the model comprising a digital representation of collection of atoms each with radius and electric charge;
defining a surface that surrounds the molecule of the model and that corresponds to a boundary of solvent contact with the molecule, and partitioning the surface using discrete surface elements, wherein the surface elements provide a non-spherical surface on the molecule;
defining a plurality of field points in the solvent near the surface elements of the molecule;
using the model, surface elements and field points to compute a distribution of polarization charge induced on the surface by an electric field corresponding to interaction of the solvent with the electric field and consequent polarization of the solvent;
measuring one or more mechanical effects on the molecule due caused by the polarization charge and by pressure exerted by the polarization charge.
generating a visual representation of the distribution of the polarization charge induced on the surface by the electric field corresponding to the interaction of the solvent with the electric field and consequent polarization of the solvent; and
causing a display device to output the visual representation.

13. The computer program product of claim 12, wherein:
the programming instructions to partition the surface comprise instructions to define the surface elements to include or neighbor one or more of the atoms of the molecule; and
the programming instructions to measure the one or more mechanical effects comprise instructions to measure a direct through-space force at each of the atoms due to the electric field caused by the induced distribution of polarization charge acting upon a charge associated with the atom.

14. The computer program product of claim 12, wherein the programming instructions to provide the digital computer simulation further comprise instructions to measure a pressure exerted by polarization density defined on the surface elements.

15. The computer program product of claim 12, wherein the programming instructions to define the field points comprise instructions to derive the field points from one or more of the following:
spherical probes that are offset from and in contact with the surface;
a regular grid exterior to the surface; or
a combination of the spherical probes and the regular grid around the molecule.

16. The computer program product of claim 12, wherein the programming instructions to measure the pressure exerted by the polarization charge comprise instructions to, for each surface element, use a probe offset from the surface element to apply a pressure vector to the surface element, wherein the probe transfers contact forces to the atoms of the collection of atoms that the probe contacts.

17. The computer program product of claim 12, wherein the programming instructions further comprise instructions to measure total pressure exerted by the polarization charge on the atoms, by adding individual contact forces exerted on individual atoms by pressure of the surface elements on the field points.

18. The computer program product of claim 12, wherein:
the field points comprise a grid; and
the programming instructions to define the grid comprise instructions to:
define a series of voxelized volume elements exterior to the surface of the molecule;
provide grid points at centers of the voxelized volume elements; and
generate a layer of the grid points around the molecule using volume elements that adjoin the surface.

19. A system comprising:
a processor;
a display device; and
a memory device that stores programming instructions that are configured to cause the processor to provide a digital computer simulation of effects of a solvent on a molecule by:
accessing a model of a molecule, the model comprising a digital representation of collection of atoms each with radius and electric charge,
defining a surface that surrounds the molecule of the model and that corresponds to a boundary of solvent contact with the molecule, and partitioning the surface using discrete surface elements, wherein the surface elements provide a non-spherical surface on the molecule,
defining a plurality of field points in the solvent near the surface elements of the molecule,;
using the model, surface elements and field points to compute a distribution of polarization charge induced on the surface by an electric field corresponding to interaction of the solvent with the electric field and consequent polarization of the solvent,
measuring one or more mechanical effects on the molecule due caused by the polarization charge and by pressure exerted by the polarization charge,
generating a visual representation of the distribution of the polarization charge induced on the surface by the electric field corresponding to the interaction of the solvent with the electric field and consequent polarization of the solvent; and
causing a display device to output the visual representation.
